(19) United States
(12) Patent Application Publication
(10) Pub. No.: US 2014/0378682 A1
(43) Pub. Date: Dec. 25, 2014

(54) PROCESSES AND INTERMEDIATES FOR PREPARING RIVAROXABAN

(71) Applicants: Shriprakash Dhar Dwivedi, Ahmedabad (IN); Ashok Prasad, Ahmedabad (IN); Daya Ram Pal, Ahmedabad (IN); Mukul Hari Prasad Sharma, Ahmedabad (IN); Kuldeep Natwarlal Jain, Ahmedabad (IN); Naitik Bharatbhai Patel, Ahmedabad (IN)

(72) Inventors: Shriprakash Dhar Dwivedi, Ahmedabad (IN); Ashok Prasad, Ahmedabad (IN); Daya Ram Pal, Ahmedabad (IN); Mukul Hari Prasad Sharma, Ahmedabad (IN); Kuldeep Natwarlal Jain, Ahmedabad (IN); Naitik Bharatbhai Patel, Ahmedabad (IN)

(73) Assignee: CADILA HEALTHCARE LIMITED, Ahmedabad, Gujarat (IN)

(21) Appl. No.: 14/343,785

(22) PCT Filed: Sep. 10, 2012
(86) PCT No.: PCT/IN2012/000599

§ 371 Date: Sep. 11, 2014

(30) Foreign Application Priority Data
Sep. 8, 2011 (IN) 2509/MUM/2011
Sep. 15, 2011 (IN) 2621/MUM/2011
Feb. 7, 2012 (IN) 0348/MUM/2012
May 7, 2012 (IN) 1403/MUM/2012

(51) Int. Cl. C07D 413/14; C07D 413/10; C07D 265/32

(52) U.S. Cl. CPC C07D 413/14; C07D 265/32; C07D 413/10
USPC 544/137; 544/166

(57) ABSTRACT

The invention discloses processes for the preparation of rivaroxaban and its pharmaceutically acceptable salts, solvates, and hydrates thereof. The invention also relates to novel intermediates for the preparation of rivaroxaban.

### FIELD OF THE INVENTION

The invention relates to processes for the preparation of rivaroxaban and its pharmaceutically acceptable salts, solvates, and hydrates thereof. The invention also relates to novel intermediates for the preparation of rivaroxaban.

### BACKGROUND OF THE INVENTION

Rivaroxaban is the INN of the anticoagulant compound (S)-5-chloro-N-{[(5S)-2-Oxo-3-[4-(3-oxo-morpholin-4-yl)phenyl]oxazolidin-5-yl]methylthiophene-2-carboxamide, represented by Formula (1)

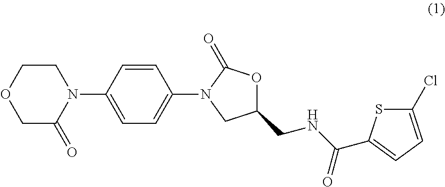 [SMILES: O=C(NC[C@H]1CN(c2ccc(N3CCOCC3=O)cc2)C(=O)O1)c1ccc(Cl)s1]

Rivaroxaban is a small molecule inhibitor of blood coagulation factor Xa and is used in the pro-phylaxis and treatment of thromboembolic diseases such as heart attack, angina pectoris, reocclusion and restenosis following angioplasty or bypass, cerebral apoplexy, transient ischemic attack, peripheral arterial obstructive diseases, pulmonary embolism and venous thrombosis.

A process for the preparation of rivaroxaban and intermediates was originally disclosed in PCT Publication No. WO 01/47919 A1.

This process discloses various disadvantages in the reaction management which has particularly unfavorable effects for preparation of the rivaroxaban on the industrial scale. Furthermore, rivaroxaban is purified by “tedious chromatographic purification”, i.e. by flash-chromatography from mixture of dichloromethane and methanol.

Similar process is described also in Journal of Medicinal Chemistry, 2005, 48, 5900-5908.

PCT Publication No. WO 2004/060887 A1 discloses a method for producing Rivaroxaban from 5-Chloro thiophene-2-carbonyl chloride, (2S)-3-amino-propane-1,2-diol and 4-(4-aminophenyl)-3-morpholinone.

This synthesis uses toxic solvents or reagents, which is disadvantageous per se, and in addition these toxic substances must be removed from the final product for regulatory reasons, which signifies additional expense. According to description the product is obtained by precipitation and filtration after cooling the reaction mixture toluene/l-methyl-2-pyrolidone, further by washing with water and drying.

PCT Publication No. WO2005/068456 A1 describes the process for purification of Rivaroxaban.

PCT Publication No. WO2007/039132 A1 discloses preparation of alternative forms, such as amorphous form, polymorphic form II and III. Further modifications such as hydrate, NMP solvate and inclusion compound with THF are also disclosed in the same document.

Rivoroxaban obtained according to PCT Publication No. WO 01/47919 A1 has crystal modification which was designated as modification I and has a melting point of 232 to 233° C.

Characteristic X-ray diffractograms of form I, II and III, hydrate and NMP solvate, are for example disclosed in PCT Publication No. WO 2007/039132 A1. The same document discloses IR spectrum, Raman spectrum and NIR spectrum of the same forms.

However, there are several drawbacks associated with the processes described in the art. These drawbacks include the use of tedious chromatography for purification which may not be feasible on a commercial scale, use of toxic substances during the reaction and low yields of the product. Because the prior art processes do not efficiently remove certain impurities, there is a need for improved process for preparing rivaroxaban.

### SUMMARY OF THE INVENTION

In one general aspect there is provided a process for preparing rivaroxaban of Formula (1) or its pharmaceutically acceptable salts, solvates, and hydrates thereof.

In another general aspect there are provided novel intermediates for the preparation of rivaroxaban.

In another general aspect there is provided a novel intermediate compound, (R)-2-hydroxy-3-(4-(3-oxo-morpholino)phenylamino)propyl ester of Formula (EE), wherein R is C₁-C₅ alkyl.

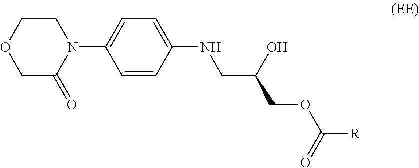

In another general aspect there is provided a novel intermediate compound, (R)-(2-oxo-3-(4-(3-oxo-morpholino)phenyl)oxazolidin-5-yl) methyl ester of Formula (FF), wherein R is C₁-C₅ alkyl.

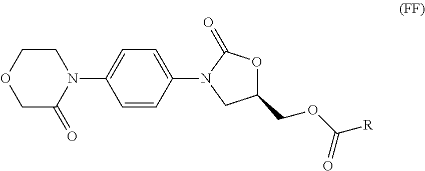

In another general aspect there is provided a novel compound of Formula (HH), wherein R₁ represents C₁-C₅ alkyl or substituted or unsubstituted aryl.

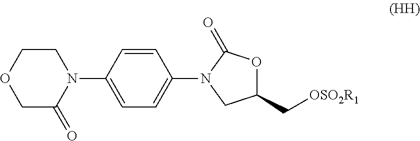

In another general aspect there is provided a novel intermediate compound, (R)-2-hydroxy-3-(4-(3-oxo-morpholino)phenylamino)propyl butyrate of Formula (E′).

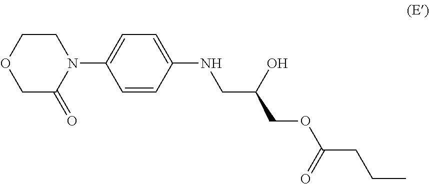 [SMILES: CCCC(=O)OC[C@H](O)CNc1ccc(N2CCOCC2=O)cc1]

In another general aspect there is provided a solid state form of the novel intermediate compound, (R)-2-hydroxy-3-(4-(3-oxo-morpholino)phenylamino)propyl butyrate of Formula (E′), which is characterized by XRD and IR.

In another general aspect there is provided a novel intermediate compound, (R)-(2-oxo-3-(4-(3-oxo-morpholino)phenyl)oxazolidin-5-yl)methyl butyrate of Formula (F′).

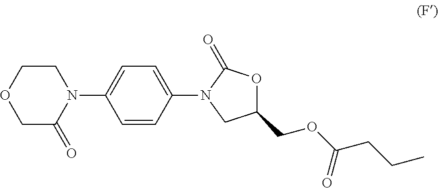 [SMILES: CCCC(=O)OC[C@H]1CN(c2ccc(N3CCOCC3=O)cc2)C(=O)O1]

In another general aspect there is provided a solid state form of the novel intermediate compound, (R)-(2-oxo-3-(4-(3-oxo-morpholino)phenyl)oxazolidin-5-yl)methyl butyrate of Formula (F′), which is characterized by XRD and IR.

In another general aspect there is provided a solid state form of (R)-4-(4-(5-(hydroxymethyl)-2-oxo-oxazolidin-3-yl)phenyl)morpholin-3-one of Formula (G), which is characterized by XRD and IR.

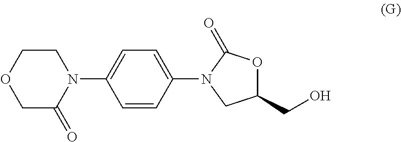 [SMILES: O=C1COCCN1c1ccc(N2C[C@H](CO)OC2=O)cc1]

In another general aspect there is provided a solid state form of (R)-(2-oxo-3-(4-(3-oxo-morpholino)phenyl)oxazolidin-5-yl)methyl 4-methylbenzenesulfonate of Formula (H), which is characterized by XRD and IR as depicted in FIG. 7 and FIG. 8, respectively.

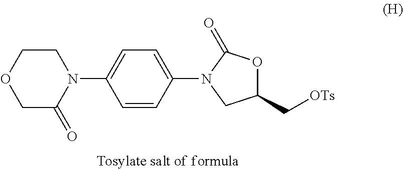 [SMILES: Cc1ccc(S(=O)(=O)OC[C@H]2CN(c3ccc(N4CCOCC4=O)cc3)C(=O)O2)cc1]

In another general aspect there is provided a solid state form of (S)-4-(4-(5-(aminomethyl)-2-oxo-oxazolidin-3-yl)phenyl)morpholin-3-one of Formula (J), which is characterized by XRD and IR.

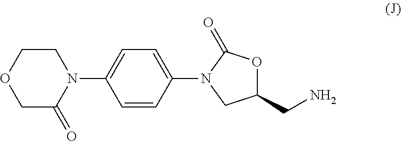 [SMILES: NC[C@H]1CN(c2ccc(N3CCOCC3=O)cc2)C(=O)O1]

In another general aspect there is provided the use of novel intermediates for the preparation of rivaroxaban of Formula (1).

In another general aspect there is provided an improved process for the preparation of key intermediates of rivaroxaban. The invention provides a process for the preparation of 4-(4-aminophenyl)morpholin-3-one compound of Formula (C) and (S)-2-(oxiran-2-ylmethyl)isoindoline-1,3-dione of Formula (E) as shown below:

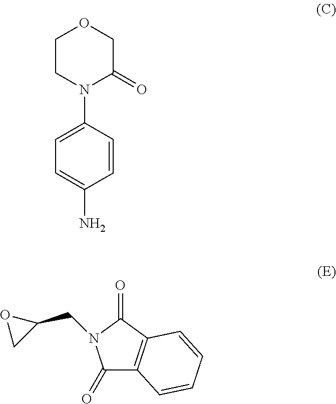 [SMILES: Nc1ccc(N2CCOCC2=O)cc1 ; O=C1c2ccccc2C(=O)N1C[C@H]1CO1]

In another general aspect there is provided a solid state form of 4-(4-aminophenyl) morpholin-3-one of Formula (C), which is characterized by XRD and DSC.

In another general aspect there is provided a process for the preparation of a solid state form of 4-(4-aminophenyl)morpholin-3-one of Formula (C), the process comprising:

i) reacting 2-(phenylamino)ethanol with 2-chloroacetyl chloride in a suitable solvent in the presence of a base to obtain 4-phenylmorpholin-3-one of Formula (A);
ii) reacting 4-phenylmorpholin-3-one of Formula (A) with a nitrating agent to obtain 4-(4-nitro phenyl) morpholin-3-one of Formula (B); and
iii) hydrogenating 4-(4-nitrophenyl)morpholin-3-one of Formula (B) in the presence of a hydrogenation catalyst in a halogenated solvent to obtain 4-(4-aminophenyl) morpholin-3-one compound of formula (C).

In another general aspect there is provided a process for the preparation of (5)-2-(oxiran-2-ylmethyl)isoindoline-1,3-dione of Formula (E):

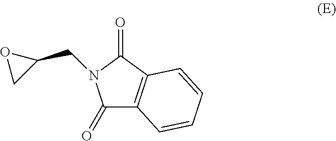 [SMILES: O=C1c2ccccc2C(=O)N1C[C@H]1CO1]

In one general aspect there is provided acid addition salts of (S)-4-(4-(5-(aminomethyl)-2-oxo-oxazolidin-3-yl)phenyl)morpholin-3-one of Formula (J1),

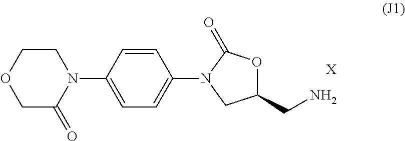

wherein X represents an inorganic acid or an organic acid.

In another general aspect there is provided solid state forms of acid addition salts of (S)-4-(4-(5-(aminomethyl)-2-oxo-oxazolidin-3-yl)phenyl)morpholin-3-one of Formula (J1)

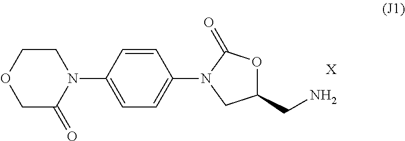

having purity greater than about 99%, specifically greater than about 99.8% as measured by HPLC.

In another general aspect there is provided the use of solid state forms of acid addition salts of (S)-4-(4-(5-(aminomethyl)-2-oxo-oxazolidin-3-yl)phenyl)morpholin-3-one of Formula (J1) for the preparation of rivaroxaban of Formula (1).

In another general aspect there is provided a process for the preparation acid addition salts of (S)-4-(4-(5-(aminomethyl)-2-oxo-oxazolidin-3-yl)phenyl)morpholin-3-one of Formula (J1), the process comprising:
(i) providing a solution of (S)-4-(4-(5-(aminomethyl)-2-oxo-oxazolidin-3-yl)phenyl)morpholin-3-one of formula (J) in a suitable solvent;
(ii) treating with an inorganic acid or an organic acid; and
(iii) obtaining the acid addition salts of (S)-4-(4-(5-(aminomethyl)-2-oxo-oxazolidin-3-yl)phenyl)morpholin-3-one of formula (J1).

In another general aspect there is provided a solid state form of the formate salt of (S)-4-(4-(5-(aminomethyl)-2-oxo-oxazolidin-3-yl)phenyl)morpholin-3-one of Formula (JF),

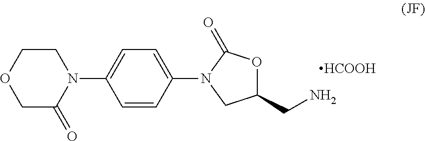 [SMILES: NC[C@H]1CN(c2ccc(N3CCOCC3=O)cc2)C(=O)O1 ; "·HCOOH"]

which is characterized by X-ray powder diffraction (XRD), DSC and IR as depicted in FIG. 13, FIG. 14 and FIG. 15, respectively.

In another general aspect there is provided a process for the preparation of a solid state form of the formate salt of (S)-4-(4-(5-(aminomethyl)-2-oxo-oxazolidin-3-yl)phenyl) morpholin-3-one of Formula (JF).

In another general aspect there is provided a solid state form of the oxalate salt of (S)-4-(4-(5-(aminomethyl)-2-oxo-oxazolidin-3-yl)phenyl)morpholin-3-one of Formula (JO),

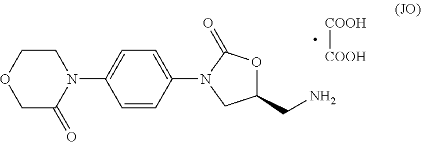

which is characterized by X-ray powder diffraction (XRD) as depicted in FIG. 16.

In another general aspect there is provided a process for the preparation of a solid state form of the oxalate salt of (S)-4-(4-(5-(aminomethyl)-2-oxo-oxazolidin-3-yl)phenyl)morpholin-3-one of Formula (JO).

In another general aspect there is provided a solid state form of the succinate salt of (S)-4-(4-(5-(aminomethyl)-2-oxo-oxazolidin-3-yl)phenyl)morpholin-3-one of Formula (JS)

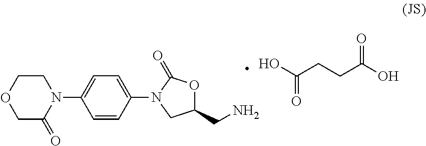 [SMILES: NC[C@H]1CN(c2ccc(N3CCOCC3=O)cc2)C(=O)O1 ; O=C(O)CCC(=O)O]

which is characterized by X-ray powder diffraction (XRD) as depicted in FIG. 17.

In another general aspect there is provided a process for the preparation of a solid state form of the succinate salt of (S)-4-(4-(5-(aminomethyl)-2-oxo-oxazolidin-3-yl)phenyl)morpholin-3-one of Formula (JS).

In another general aspect there is provided a solid state form of the mandelate salt of (S)-4-(4-(5-(aminomethyl)-2-oxo-oxazolidin-3-yl)phenyl)morpholin-3-one of Formula (JM),

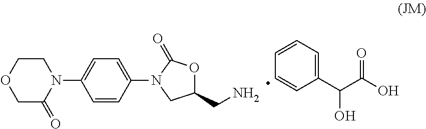 [SMILES: NC[C@H]1CN(c2ccc(N3CCOCC3=O)cc2)C(=O)O1 ; O=C(O)C(O)c1ccccc1]

which is characterized by X-ray powder diffraction (XRD) as depicted in FIG. 18.

In another general aspect there is provided a process for the preparation of a solid state form of the L(+)-mandelate salt of (S)-4-(4-(5-(aminomethyl)-2-oxo-oxazolidin-3-yl)phenyl) morpholin-3-one of Formula (JM).

In another general aspect there is provided a process for preparing rivaroxaban of Formula (1) or its pharmaceutically acceptable salts, solvates, and hydrates thereof,

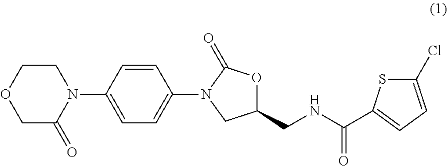 [SMILES: O=C(NC[C@H]1CN(c2ccc(N3CCOCC3=O)cc2)C(=O)O1)c1ccc(Cl)s1]

the process comprising:
(i) reacting 4-(4-aminophenyl)morpholin-3-one compound of Formula (C) with (R)-glycidyl alkyl ester of Formula (E1), wherein R represents C₁-C₅ alkyl, in a suitable solvent to obtain (R)-2-hydroxy-3-(4-(3-oxomorpholino)phenylamino)alkyl ester of Formula (EE);

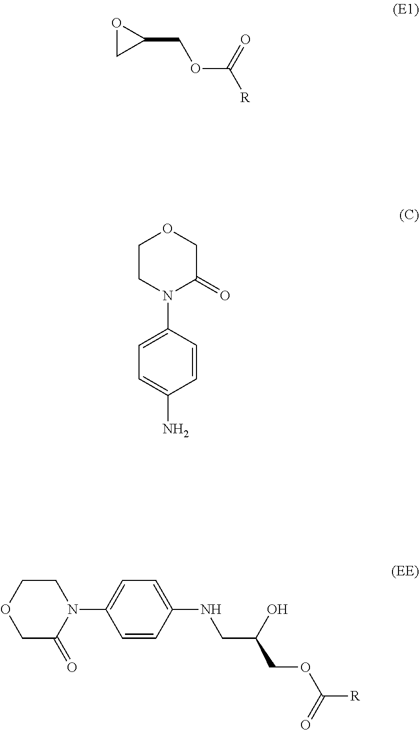

(ii) reacting (R)-2-hydroxy-3-(4-(3-oxomorpholino)phenylamino)alkyl ester of Formula (EE) with a cyclizing agent in a suitable solvent, optionally in the presence of a catalyst to obtain (R)-(2-oxo-3-(4-(3-oxomorpholino)phenyl)oxazolidin-5-yl)alkyl ester of Formula (FF);

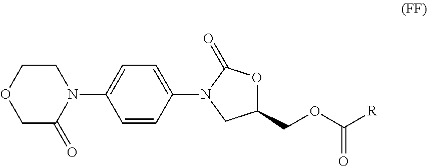

(iii) reacting (R)-(2-oxo-3-(4-(3-oxomorpholino)phenyl)oxazolidin-5-yl)alkyl ester of Formula (FF) with a base to obtain (R)-4-(4-(5-(hydroxymethyl)-2-oxo-oxazolidin-3-yl)phenyl)morpholin-3-one of Formula (GG);

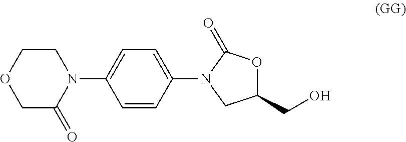 [SMILES: O=C1COCCN1c1ccc(N2C[C@H](CO)OC2=O)cc1]

(iv) reacting (R)-4-(4-(5-(hydroxymethyl)-2-oxo-oxazolidin-3-yl)phenyl)morpholin-3-one of Formula (GG) with a compound of formula (2), wherein R₁ represents C₁-C₅ alkyl or substituted or unsubstituted aryl, and X represents halide selected from Br, Cl, F or I,

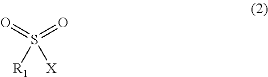

in the presence of a base to obtain a compound of formula (HH);

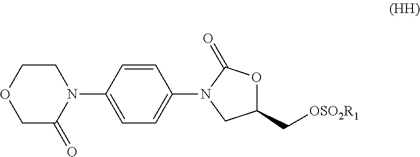

(v) reacting the compound of formula (HH) with phthalimide, optionally in the presence of a base to obtain (S)-2-((2-oxo-3-(4-(3-oxo-morpholino)phenyl)oxazolidin-5-yl)methyl)isoindoline-1,3-dione of Formula (I);

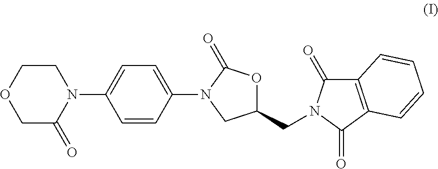 [SMILES: O=C1c2ccccc2C(=O)N1C[C@H]1CN(c2ccc(N3CCOCC3=O)cc2)C(=O)O1]

(vi) reacting (S)-2-((2-oxo-3-(4-(3-oxo-morpholino)phenyl)oxazolidin-5-yl)methyl)isoindoline-1,3-dione of Formula (I) with a base in a suitable solvent to obtain (S)-4-(4-(5-(aminomethyl)-2-oxooxazolidin-3-yl)phenyl)morpholin-3-one (J); and

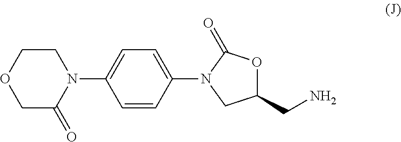 [SMILES: NC[C@H]1CN(c2ccc(N3CCOCC3=O)cc2)C(=O)O1]

(vii) converting the (S)-4-(4-(5-(aminomethyl)-2-oxooxazolidin-3-yl)phenyl)morpholin-3-one (J) to rivaroxaban of Formula (1).

In another general aspect there is provided an improved process for preparing rivaroxaban of Formula (1) or pharmaceutically acceptable salts, solvates, and hydrates thereof,

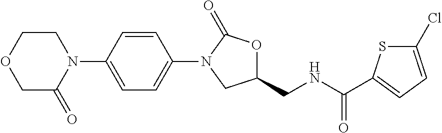 [SMILES: O=C(NC[C@H]1CN(c2ccc(N3CCOCC3=O)cc2)C(=O)O1)c1ccc(Cl)s1]

the process comprising:
i) reacting 4-(4-aminophenyl)morpholin-3-one of Formula (C) with (S)-2-(oxiran-2-yl methyl)isoindoline-1,3-dione of Formula (E) in a suitable solvent in the absence of a base to obtain (R)-2-(2-hydroxy-3-(4-(3-oxo-morpholino)phenylamino)propyl)isoindoline-1,3-dione of Formula (F);
ii) reacting (R)-2-(2-hydroxy-3-(4-(3-oxo-morpholino)phenylamino)propyl) isoindoline-1,3-dione of formula (F) with a cyclizing agent in a suitable solvent in the presence of a catalyst to obtain (S)-2-((2-oxo-3-(4-(3-oxo-morpholino)phenyl) oxazolidin-5-yl)methyl)isoindoline-1,3-dione of formula (I);
iii) reacting(S)-2-((2-oxo-3-(4-(3-oxo-morpholino)phenyl)oxazolidin-5-yl)methyl) isoindoline-1,3-dione of formula (I) in the presence of a base in a suitable solvent to obtain (S)-4-(4-(5-(aminomethyl)-2-oxo-oxazolidin-3-yl)phenyl)morpholin-3-one (J); and
iv) reacting the (S)-4-(4-(5-(aminomethyl)-2-oxo-oxazolidin-3-yl)phenyl)morpholin-3-one of formula (J) with 5-chlorothiophene-2-carboxylic acid chloride in a biphasic solvent system in the presence of a base to obtain rivaroxaban.

In another general aspect there is provided a chiral pure (S)-2-((2-oxo-3-(4-(3-oxo-morpholine) phenyl)oxazolidin-5-yl)methyl)isoindoline-1,3-dione of Formula (I) having chiral purity greater than about 99%, specifically greater than about 99.5%, more specifically greater than about 99.9% and most specifically greater than about 99.98% as measured by HPLC.

In another general aspect there is provided an improved process for the enrichment of chiral purity of (S)-2-((2-oxo-3-(4-(3-oxo-morpholino)phenyl)oxazolidin-5-yl)methyl)isoindoline-1,3-dione of formula (I),

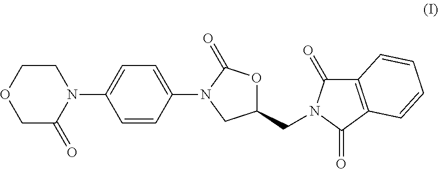 [SMILES: O=C1c2ccccc2C(=O)N1C[C@H]1CN(c2ccc(N3CCOCC3=O)cc2)C(=O)O1]

the process comprising:
i) crystallizing (S)-2-(2-oxo-3-(4-(3-oxo-morpholino)phenyl)oxazolidin-5-yl)methyl) isoindoline-1,3-dione of formula (I) from one or more organic solvents; and
ii) isolating the chirally pure (S)-2-((2-oxo-3-(4-(3-oxo-morpholino)phenyl)oxazolidin-5-yl)methyl)isoindoline-1,3-dione of Formula (I).

In another general aspect there is provided rivaroxaban of Formula (1) substantially free of impurity X, i.e. (R)-5-chloro-N-(2-hydroxy-3-(4-(3-oxo-morpholino)phenylamino) propyl)thiophene-2-carboxamide of Formula (X).

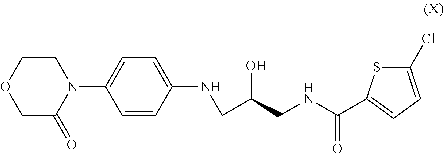 [SMILES: O=C(NC[C@H](O)CNc1ccc(N2CCOCC2=O)cc1)c1ccc(Cl)s1]

In another general aspect there is provided rivaroxaban of Formula (1) substantially free of impurity Y, i.e. (S)-4-(4-(3-amino-2-hydroxypropylamino)phenyl)morpholin-3-one of Formula (Y).

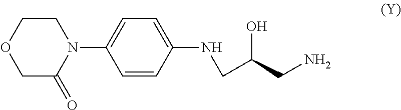 [SMILES: NC[C@H](O)CNc1ccc(N2CCOCC2=O)cc1]

In another general aspect there is provided rivaroxaban of Formula (1) having a total purity of greater than about 99%, specifically greater than about 99.5%, more specifically greater than about 99.9%, and most specifically greater than about 99.98% as measured by HPLC.

In another general aspect there is provided micronized rivaroxaban having a particle size in terms of d(90) less than about 100 microns.

In another general aspect there is provided micronized rivaroxaban having a particle size in terms of d(90) less than about 50 microns.

In another general aspect there is provided microcrystalline rivaroxaban having a particle size in terms of d(90) less than about 10 microns.

In another general aspect there is provided a rivaroxaban of Formula (1) having a chiral purity of greater than about 99%, specifically greater than about 99.5%, more specifically greater than about 99.9%, and most specifically greater than about 99.98%.

### BRIEF DESCRIPTION OF THE ACCOMPANYING DRAWINGS

FIG. 1 is a characteristic powder X-ray diffraction (XRD) pattern of (R)-2-hydroxy-3-(4-(3-oxo-morpholino)phenylamino)propyl butyrate of Formula (E′).

FIG. 2 is a characteristic infra-red (IR) spectrum of (R)-2-hydroxy-3-(4-(3-oxo-morpholino)phenylamino)propyl butyrate of Formula (E′).

FIG. 3 is a characteristic powder X-ray diffraction (XRD) pattern of (R)-(2-oxo-3-(4-(3-oxo-morpholino)phenyl)oxazolidin-5-yl)methyl butyrate of Formula (F′).

FIG. 4 is a characteristic infra-red (IR) spectrum of (R)-(2-oxo-3-(4-(3-oxo-morpholino)phenyl)oxazolidin-5-yl)methyl butyrate of Formula (F′).

FIG. 5 is a characteristic powder X-ray diffraction (XRD) pattern of (R)-4-(4-(5-(hydroxymethyl)-2-oxo-oxazolidin-3-yl)phenyl)morpholin-3-one of Formula (GG).

FIG. 6 is a characteristic infra-red (IR) spectrum of (R)-4-(4-(5-(hydroxymethyl)-2-oxo-oxazolidin-3-yl)phenyl)morpholin-3-one of Formula (GG).

FIG. 7 is a characteristic powder X-ray diffraction (XRD) pattern of (R)-(2-oxo-3-(4-(3-oxo-morpholino)phenyl)oxazolidin-5-yl)methyl 4-methylbenzenesulfonate of Formula (H).

FIG. 8 is a characteristic infra-red (IR) spectrum of (R)-(2-oxo-3-(4-(3-oxo-morpholino)phenyl)oxazolidin-5-yl)methyl 4-methylbenzenesulfonate of Formula (H).

FIG. 9 is a characteristic powder X-ray diffraction (XRD) pattern of (S)-4-(4-(5-(aminomethyl)-2-oxo-oxazolidin-3-yl)phenyl)morpholin-3-one of Formula (J).

FIG. 10 is a characteristic infra-red (IR) spectrum of (S)-4-(4-(5-(aminomethyl)-2-oxo-oxazolidin-3-yl)phenyl)morpholin-3-one of Formula (J).

FIG. 11. shows X-ray diffractogram (XRD) of solid state form of 4-(4-aminophenyl)morpholin-3-one of Formula (C).

FIG. 12. shows Differential Scanning calorimetry (DSC) of solid state form of 4-(4-aminophenyl)morpholin-3-one of Formula (C).

FIG. 13: shows X-ray diffractogram (XRD) of solid state form of a formate salt of (S)-4-(4-(5-(aminomethyl)-2-oxo-oxazolidin-3-yl)phenyl) morpholin-3-one of Formula (JF).

FIG. 14: shows Differential Scanning calorimetry (DSC) of solid state form of a formate salt of (S)-4-(4-(5-(aminomethyl)-2-oxo-oxazolidin-3-yl)phenyl)morpholin-3-one of Formula (JF).

FIG. 15: shows Infra-red (IR) of solid state form of a formate salt of (S)-4-(4-(5-(aminomethyl)-2-oxo-oxazolidin-3-yl)phenyl)morpholin-3-one of Formula (JF).

FIG. 16: shows X-ray diffractogram (XRD) of solid state form of a oxalate salt of (S)-4-(4-(5-(aminomethyl)-2-oxo-oxazolidin-3-yl)phenyl)morpholin-3-one of Formula (JO).

FIG. 17: shows X-ray diffractogram (XRD) of solid state form of a succinate salt of (S)-4-(4-(5-(aminomethyl)-2-oxo-oxazolidin-3-yl)phenyl)morpholin-3-one of Formula (JS).

FIG. 18: shows X-ray diffractogram (XRD) of solid state form of a mandelate salt of (S)-4-(4-(5-(aminomethyl)-2-oxo-oxazolidin-3-yl)phenyl)morpholin-3-one of Formula (JM).

### DETAILED DESCRIPTION OF THE INVENTION

In one general aspect there is provided a new process for the preparation of rivaroxaban of Formula (1) or its pharmaceutically acceptable salts, solvates, and hydrates thereof.

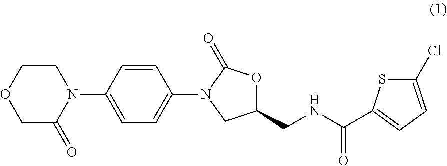 [SMILES: O=C(NC[C@H]1CN(c2ccc(N3CCOCC3=O)cc2)C(=O)O1)c1ccc(Cl)s1]

In another general aspect there is provided a process for preparing rivaroxaban of Formula (1) or its pharmaceutically acceptable salts, solvates, and hydrates thereof,

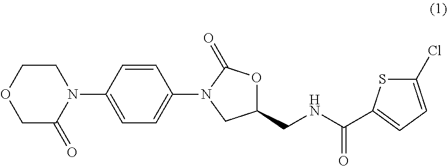 [SMILES: O=C(NC[C@H]1CN(c2ccc(N3CCOCC3=O)cc2)C(=O)O1)c1ccc(Cl)s1]

the process comprising:
(i) reacting 4-(4-aminophenyl)morpholin-3-one compound of Formula (C) with (R)-glycidyl alkyl ester of formula (E1), wherein R represents C₁-C₅ alkyl, in a suitable solvent to obtain (R)-2-hydroxy-3-(4-(3-oxo-morpholino)phenylamino)alkyl ester of formula (EE);

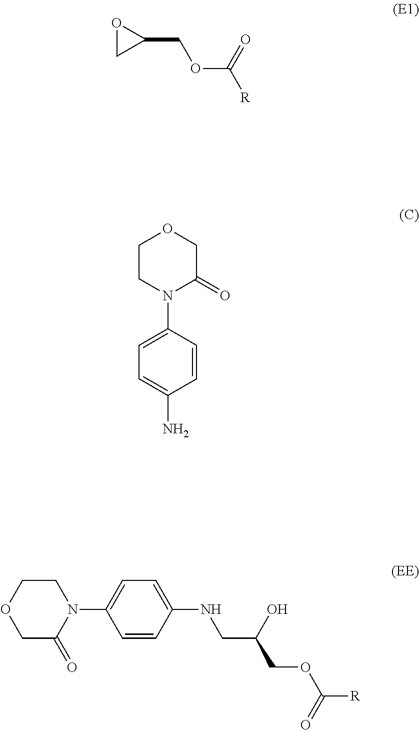

(ii) reacting (R)-2-hydroxy-3-(4-(3-oxo-morpholino)phenylamino)alkyl ester of formula (EE) with a cyclizing agent in a suitable solvent, optionally in the presence of a catalyst to obtain (R)-(2-oxo-3-(4-(3-oxo-morpholino)phenyl)oxazolidin-5-yl)alkyl ester of formula (FF);

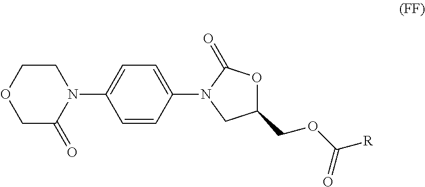

(iii) reacting (R)-(2-oxo-3-(4-(3-oxo-morpholino)phenyl)oxazolidin-5-yl)alkyl ester of formula (FF) with a base to obtain (R)-4-(4-(5-(hydroxymethyl)-2-oxo-oxazolidin-3-yl)phenyl)morpholin-3-one of formula (GG);

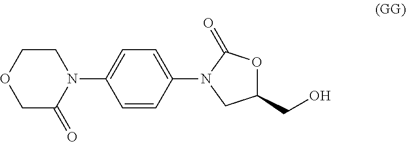 [SMILES: O=C1COCCN1c1ccc(N2C[C@H](CO)OC2=O)cc1]

(iv) reacting (R)-4-(4-(5-(hydroxymethyl)-2-oxo-oxazolidin-3-yl)phenyl)morpholin-3-one of formula (GG) with a compound of formula (2), wherein R₁ represents C₁-C₅ alkyl or substituted or unsubstituted aryl, and X represents halide selected from Br, Cl, F or I,

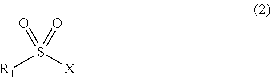

in the presence of a base to obtain a compound of formula (HH);

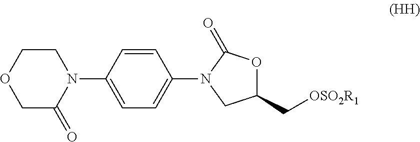

(v) reacting the compound of formula (HH) with phthalimide, optionally in the presence of a base to obtain (S)-2-((2-oxo-3-(4-(3-oxo-morpholino)phenyl)oxazolidin-5-yl)methyl)isoindoline-1,3-dione of Formula (I);

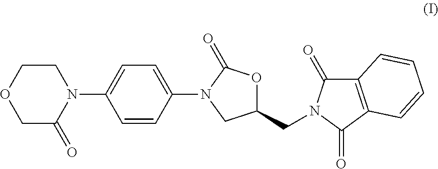 [SMILES: O=C1c2ccccc2C(=O)N1C[C@H]1CN(c2ccc(N3CCOCC3=O)cc2)C(=O)O1]

(vi) reacting (S)-2-((2-oxo-3-(4-(3-oxo-morpholino)phenyl)oxazolidin-5-yl)methyl)isoindoline-1,3-dione of formula (I) with a base in a suitable solvent to obtain (S)-4-(4-(5-(aminomethyl)-2-oxo-oxazolidin-3-yl)phenyl)morpholin-3-one (J); and

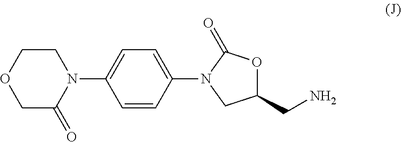 [SMILES: NC[C@H]1CN(c2ccc(N3CCOCC3=O)cc2)C(=O)O1]

(vii) converting (S)-4-(4-(5-(aminomethyl)-2-oxooxazolidin-3-yl)phenyl)morpholin-3-one (J) to rivaroxaban of Formula (1).

There is provided a process for the preparation of rivaroxaban by using novel intermediates of compounds of formula (EE), Formula (FF) and Formula (HH).

The process for preparing rivaroxaban involves reacting 4-(4-aminophenyl)morpholin-3-one compound of formula (C) with (R)-glycidyl alkyl ester of formula (E1), wherein R represents C₁-C₅ alkyl, preferably R is butyl, in a suitable solvent to obtain (R)-2-hydroxy-3-(4-(3-oxo-morpholino)phenylamino)alkyl ester of formula (EE). Suitable solvent at step (i) is selected from one or more of C₁-C₅ alcohols, esters, ethers, tetrahydrofuran (THF), water or mixtures thereof. Preferably, ethanol or ethanol-water mixture may be used for the reaction.

The (R)-2-hydroxy-3-(4-(3-oxomorpholino)phenylamino)alkyl ester of formula (EE) is cyclized by reacting with a cyclizing agent selected from N,N-carbonyldiimidazole (CDI), phosgene, and the like, in the presence of a catalyst in a suitable solvent. Suitable solvent at step (ii) may include one or more of C₁-C₅ alcohols, esters, ethers, ketones, tetrahydrofuran (THF), halogenated solvent, water or a mixture thereof.

The catalyst used in the cyclization reaction may include one or more of N,N-dimethylamino pyridine (DMAP), diisopropylamine (DIPA), diisopropyethylamine (DIPEA), and the like.

In general, the R)-(2-oxo-3-(4-(3-oxomorpholino)phenyl)oxazolidin-5-yl)alkyl ester of formula (FF) is reacted with a base in a suitable solvent to obtain (R)-4-(4-(5-(hydroxymethyl)-2-oxo-oxazolidin-3-yl)phenyl)morpholin-3-one of formula (GG). Suitable solvent at step (iii) may be selected from one or more of C₁-C₅ alcohols, esters, ethers, ketones, tetrahydrofuran (THF), halogenated solvent, water or a mixture thereof. In particular, methanol-water mixture may be used. The product is obtained in the form of a solid state form, which is further reacted with a compound of Formula (2) in the presence of a base in a suitable solvent to provide a compound of Formula (HH). In particular, the compound of Formula (GG) may be reacted with p-tosyl chloride in the presence of a base to obtain (R)-(2-oxo-3-(4-(3-oxo-morpholino)phenyl)oxazolidin-5-yl)methyl 4-methylbenzenesulfonate of Formula (H).

The base may be selected from one or more of alkali or alkaline earth metal hydroxides, alkoxides, carbonates or bicarbonates or an organic base. The base may be selected from one or more of sodium hydroxide, potassium hydroxide, sodium methoxide, potassium tert-butoxide, sodium carbonate, potassium carbonate, sodium bicarbonate, triethyl amine, diisopropyl amine, dimethyl amine, diisopropylethylamine, diisopropylmethyl amine, pyridine, piperidine, morpholine and N-methyl piperidine.

Suitable solvent at step (iv) may include one or more of C₁-C₅ alcohol, esters, ethers, ketones, tetrahydrofuran (THF), halogenated solvent, water or a mixture thereof.

Suitable solvent at step (v) may be selected from one or more of C₁-C₅ alcohol, esters, ketones, halogenated solvent, DMF, DMSO, sulfolane, water or a mixture thereof.

The base used at step (v) may include alkali or alkaline earth metal hydroxides, alkoxides, carbonates or bicarbonates selected from sodium hydroxide, potassium hydroxide, sodium methoxide, potassium tert-butoxide, sodium carbonate, potassium carbonate, and sodium bicarbonate.

In particular, the (R)-(2-oxo-3-(4-(3-oxo-morpholino)phenyl)oxazolidin-5-yl)methyl 4-methylbenzenesulfonate of Formula (H) is reacted with phthalamide in the presence of a base, for example, potassium carbonate in dimethylformamide solvent to provide (S)-2-((2-oxo-3-(4-(3-oxomorpholino) phenyl) oxazolidin-5-yl)methyl)isoindoline-1,3-dione of formula (I), which is further treated with a base selected from hydrazine hydrate, C₁-C₅ amines, for example, monomethyl amine solution in methanol to obtain (S)-4-(4-(5-(aminomethyl)-2-oxo-oxazolidin-3-yl)phenyl)morpholin-3-one of Formula (J).

Suitable solvent at step (vi) may include one or more of hydrocarbons, nitriles, amides, alcohol, ketones, ester, and the like. In particular, toluene, xylene, ethylbenzene dimethyl formamide, dimethyl acetamide, acetonitrile, C₁-C₄ straight chain or branched alcohols, acetone, methyl isobutyl ketone, methyl ethyl ketone, ethyl acetate, isopropyl acetate, butyl acetate, halogenated solvents may be used.

The invention further provides a solid state form of the acid addition salts of (S)-4-(4-(5-(aminomethyl)-2-oxo-oxazolidin-3-yl)phenyl)morpholin-3-one of formula (J1),

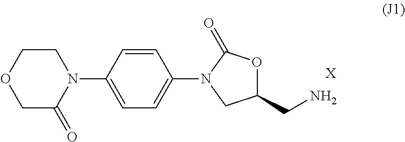

wherein X represents salts with inorganic acids or organic acids.

The acid used for the formation of salt with the compound of formula (J) may be selected from inorganic acids or organic acids. Inorganic acids such as hydrochloric acid, hydrobromic acid, phosphoric acid or sulphuric acid may be used. Organic acids such sulphonic acids, oxalic acid, formic acid, acetic acid, trifluoroacetic acid, propionic acid, maleic acid, succinic acid, fumaric acid, malic acid, citric acid, tartaric acid, lactic acid, benzoic acid, or methanesulphonic acid, mandelic acid, L(+)-mandelic acid, D(−)-mandelic acid, benzenesulphonic acid, toluenesulphonic acid or naphthalenedisulphonic acid may be used.

In particular, acids may be selected from formic acid, oxalic acid, succinic acid and L(+)-mandelic acid.

There is further provided a process for the preparation of acid addition salts of (S)-4-(4-(5-(aminomethyl)-2-oxo-oxazolidin-3-yl)phenyl)morpholin-3-one of formula (J1),

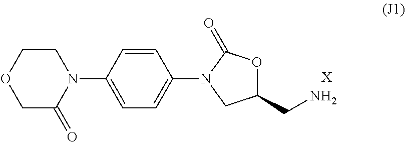

wherein X represents salts with inorganic acids or organic acids, the process comprising:
(i) providing a solution of (S)-4-(4-(5-(aminomethyl)-2-oxo-oxazolidin-3-yl)phenyl)morpholin-3-one of formula (J) in a suitable solvent;
(ii) treating with an inorganic acid or an organic acid; and
(iii) obtaining the acid addition salts of (S)-4-(4-(5-(aminomethyl)-2-oxo-oxazolidin-3-yl)phenyl)morpholin-3-one of formula (J1).

The solution of the compound of formula (J) may be obtained directly from the previous reaction mass or the compound of formula (J) may be dissolved in a solvent selected from one or more of hydrocarbons, nitriles, amides, C₁-C₅ alcohol, ketones, esters and halogenated solvents.

There is further provided a process for the purification of a compound of formula (J), the process comprising:

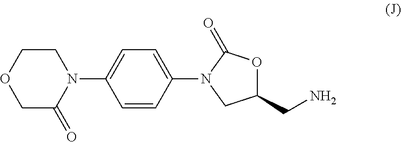 [SMILES: NC[C@H]1CN(c2ccc(N3CCOCC3=O)cc2)C(=O)O1]

(i) providing a solution of (S)-4-(4-(5-(aminomethyl)-2-oxo-oxazolidin-3-yl)phenyl)morpholin-3-one of formula (J) in a suitable solvent;
(ii) treating with an inorganic acid or an organic acid;
(iii) obtaining a solid state form of an acid addition salt of (S)-4-(4-(5-(aminomethyl)-2-oxo-oxazolidin-3-yl)phenyl)morpholin-3-one of formula (J1); and
(iv) treating the solid state form of the acid addition salt of (S)-4-(4-(5-(aminomethyl)-2-oxo-oxazolidin-3-yl)phenyl)morpholin-3-one of formula (J1) with a base in a suitable solvent to obtain pure (S)-4-(4-(5-(aminomethyl)-2-oxo-oxazolidin-3-yl)phenyl)morpholin-3-one of formula (J),

Thus, obtained pure compound of formula (J) may be converted to rivaroxaban by any known method.

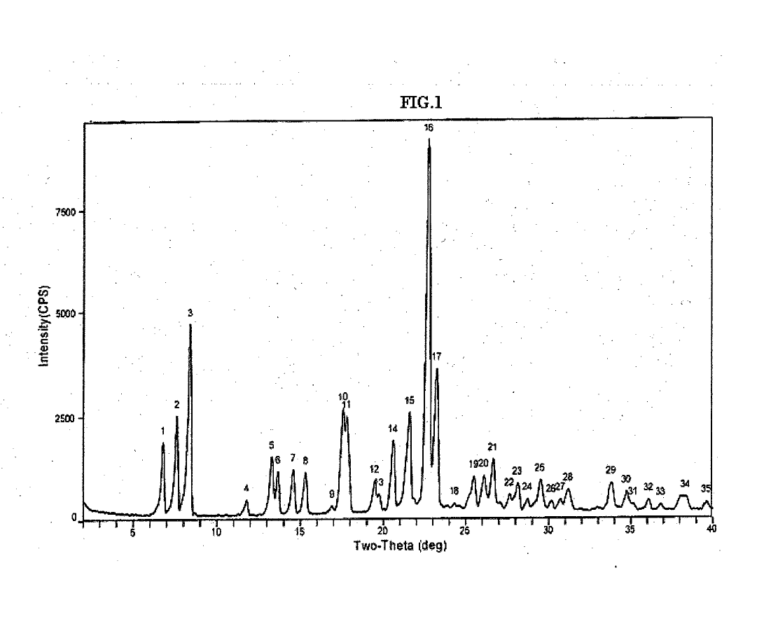

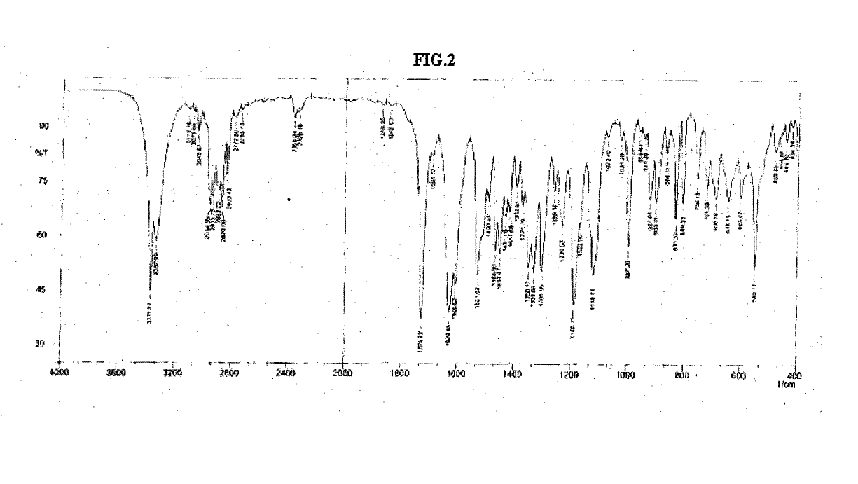

In another general aspect there is provided a solid state form of novel intermediate (R)-2-hydroxy-3-(4-(3-oxo-morpholino)phenylamino)propyl butyrate of Formula (E′), which is characterized by XRD & IR as depicted in FIG. 1 and FIG. 2 respectively.

In another general aspect there is provided a solid state form of novel intermediate compound, (R)-2-hydroxy-3-(4-(3-oxo-morpholino)phenylamino)propyl butyrate of Formula (E′),

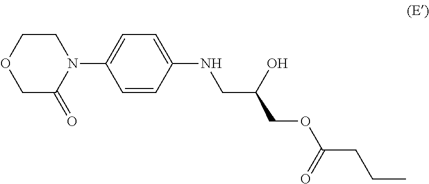 [SMILES: CCCC(=O)OC[C@H](O)CNc1ccc(N2CCOCC2=O)cc1]

which is characterized by at least one or more of the following properties:
i) a powder X-ray diffraction pattern having peaks at about 6.8, 7.6, 8.4, 13.3, 17.5, 21.5, 22.7 and ±23.2±0.2 degrees 2-theta substantially as depicted in FIG. 1;
ii) having additional peaks in XRD at 11.8, 14.5, 15.3, 17.8, 19.4, 20.6, 25.5, 26.1, 26.6, 28.1 and 29.5±0.2 degrees 2-theta;
iii) an IR spectrum substantially in accordance with FIG. 2; or
iv) an IR spectrum having absorption bands at about 3371, 3332, 3043, 2954, 2870, 2833, 2358, 1870, 1842, 1728, 1691, 1629, 1608, 1572, 1490, 1465, 1450, 1392, 1371, 1350, 1330, 1301, 1259, 1230, 1188, 1118, 1024, 997, 921, 900, 831, 806, 756, 721, 690, 646, 603 and 549±2 cm⁻¹.

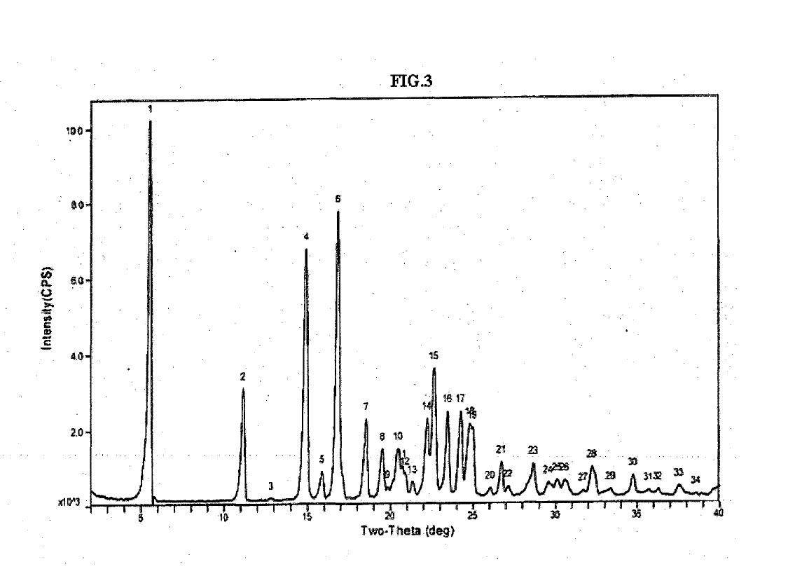

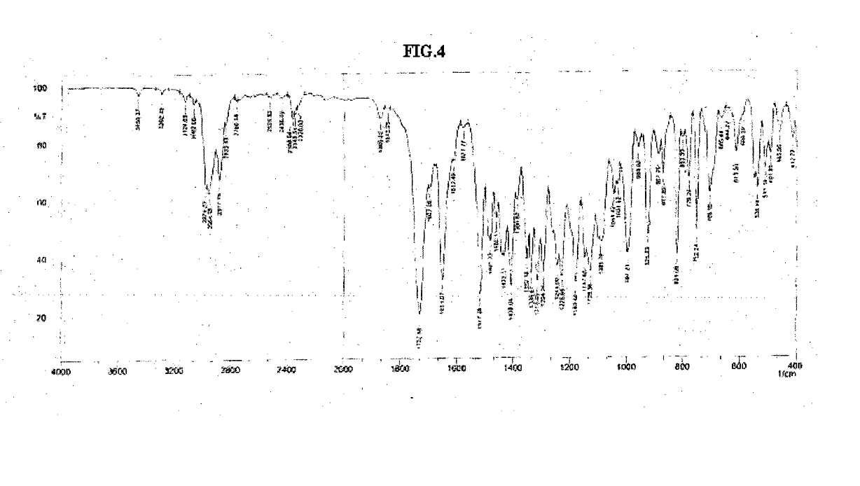

In another general aspect there is provided a solid state form of novel intermediate, (R)-(2-oxo-3-(4-(3-oxo-morpholino)phenyl)oxazolidin-5-yl)methyl butyrate of Formula (F′), which is characterized by XRD and IR as depicted in FIG. 3 and FIG. 4 respectively.

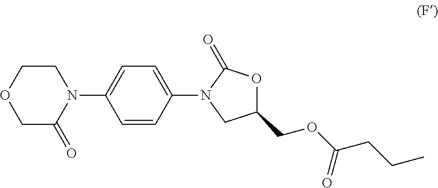 [SMILES: CCCC(=O)OC[C@H]1CN(c2ccc(N3CCOCC3=O)cc2)C(=O)O1]

A solid state form of Formula (F′) having characteristic peaks in XRD at about 5.5, 11.1, 14.9, 16.8, 18.5, 22.6, 23.4, 24.2 and 24.9±0.2 degrees 2-theta and having additional peaks at about 15.8, 19.5, 20.4, 21.3, 22.2, 26.0, 26.7, 28.6, 29.5, 30.5 and 32.2±0.2 degrees 2-theta. A solid state form of the novel intermediate (R)-(2-oxo-3-(4-(3-oxo-morpholino)phenyl) oxazolidin-5-yl)methyl butyrate of Formula (F′) having IR absorption bands at about 3458, 3292, 2974, 2954, 2877, 2358, 1869, 1732, 1651, 1517, 1481, 1386, 1352, 1336, 1315, 1294, 1180, 1128, 1089, 1045, 997, 925, 871, 821, 779, 752, 705, 665, 613, 538, 511 and 460±2 cm⁻¹.

In another general aspect there is provided a solid state form of (R)-4-(4-(5-(hydroxymethyl)-2-oxooxazolidin-3-yl)phenyl)morpholin-3-one of Formula (G),

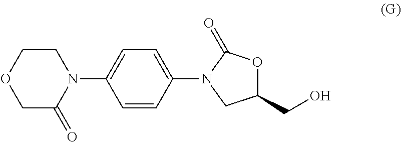 [SMILES: O=C1COCCN1c1ccc(N2C[C@H](CO)OC2=O)cc1]

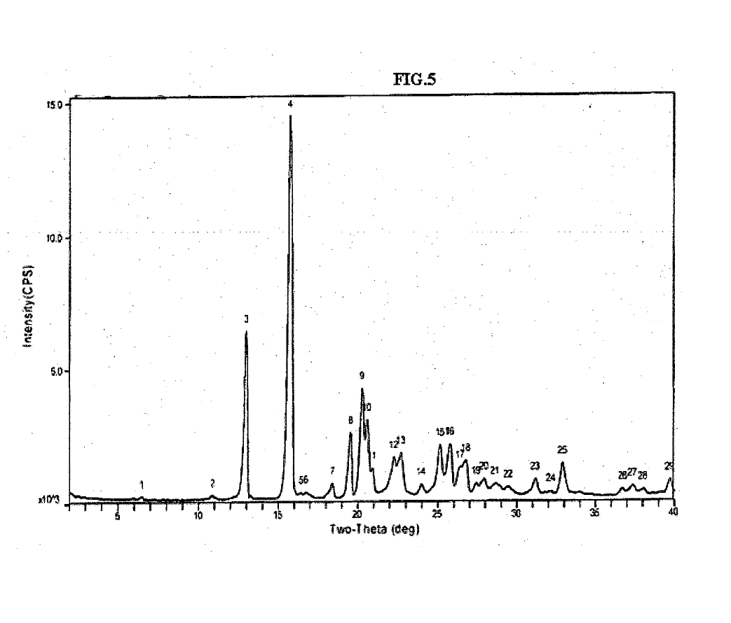

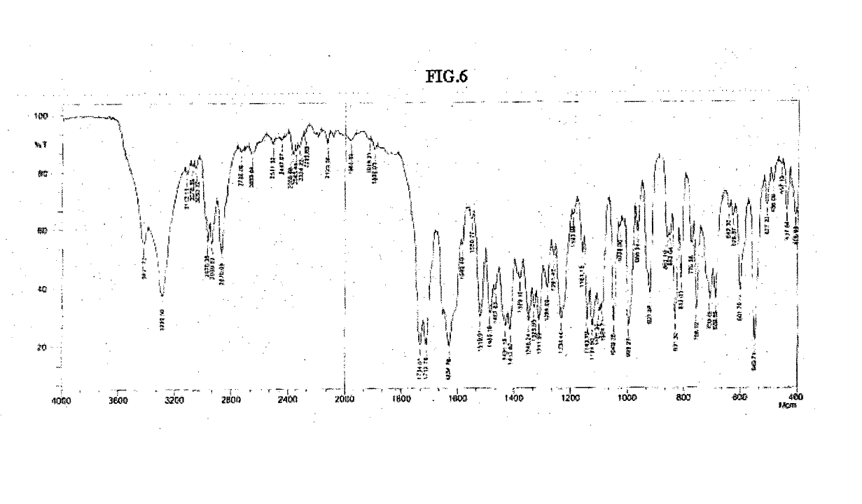

which characterized by XRD and IR as depicted in FIG. 5 and FIG. 6 respectively.

A solid state form of (R)-4-(4-(5-(hydroxymethyl)-2-oxo-oxazolidin-3-yl)phenyl)morpholin-3-one of Formula (G) having characteristic peaks in XRD at about 12.9, 15.7, 19.5, 20.2, 22.7, 25.7, 26.7 and 31.1±0.2 degrees 2-theta and additional peaks at about 6.4, 10.8, 16.4, 18.4, 20.5, 22.2, 23.9, 25.1, 27.9 and 32.9.

A solid state form of (R)-4-(4-(5-(hydroxymethyl)-2-oxo-oxazolidin-3-yl)phenyl)morpholin-3-one of Formula (G) having IR absorption bands at about 3421, 3290, 3131, 3053, 2970, 2939, 2870, 2659, 2511, 2366, 2125, 1980, 1734, 1712, 1631, 1519, 1485, 1431, 1413, 1348, 1311, 1284, 1234, 1143, 1124, 1049, 995, 921, 831, 812, 756, 709, 688, 601, 549 and 437±2 cm⁻¹.

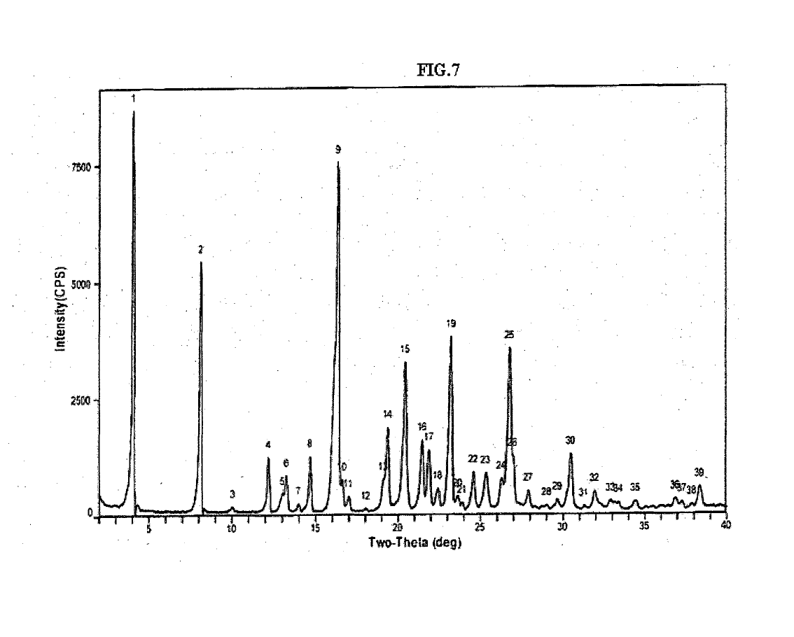

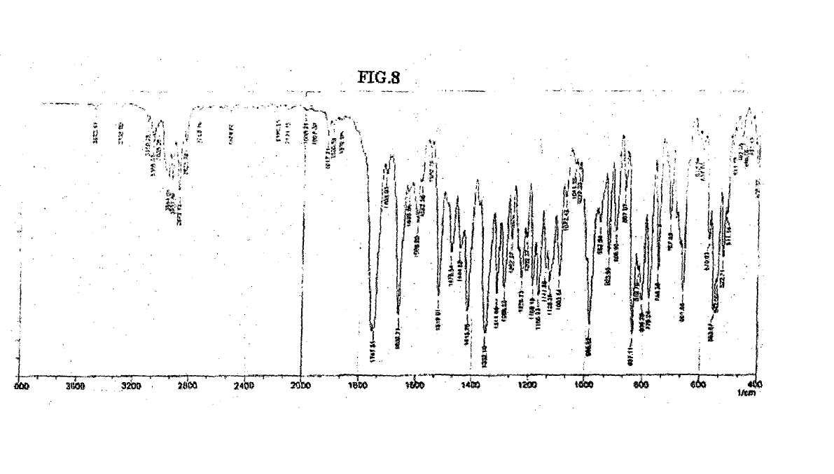

In another general aspect there is provided (R)-(2-oxo-3-(4-(3-oxomorpholino)phenyl)oxazolidin-5-yl)methyl 4-methylbenzenesulfonate of Formula (H), which is characterized by XRD and IR as depicted in FIG. 7 and FIG. 8, respectively.

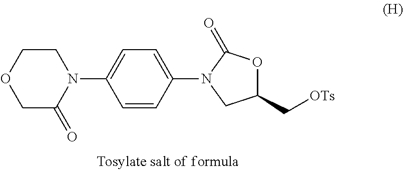 [SMILES: Cc1ccc(S(=O)(=O)OC[C@H]2CN(c3ccc(N4CCOCC4=O)cc3)C(=O)O2)cc1]

A solid state form of (R)-(2-oxo-3-(4-(3-oxo-morpholino)phenyl)oxazolidin-5-yl)methyl 4-methylbenzenesulfonate of Formula (H) having characteristic peaks in XRD at about 4.0, 8.1, 16.3, 19.3, 20.4, 23.2 and 26.7 and additional peaks at about 10.0, 12.2, 13.2, 14.6, 17.0, 21.4, 21.8, 22.4, 24.5, 25.3, 27.9 and 30.4±0.2 degrees 2-theta.

A solid state form of (R)-(2-oxo-3-(4-(3-oxo-morpholino)phenyl)oxazolidin-5-yl)methyl 4-methylbenzenesulfonate of Formula (H) having IR absorption bands at about 3483, 3305, 3066, 2954, 2879, 2742, 2524, 2121, 1917, 1747, 1660, 1598, 1519, 1475, 1415, 1352, 1311, 1286, 1226, 1188, 1166, 1128, 1093, 985, 923, 896, 837, 779, 707, 661, 553, 522 and 460±2 cm⁻¹.

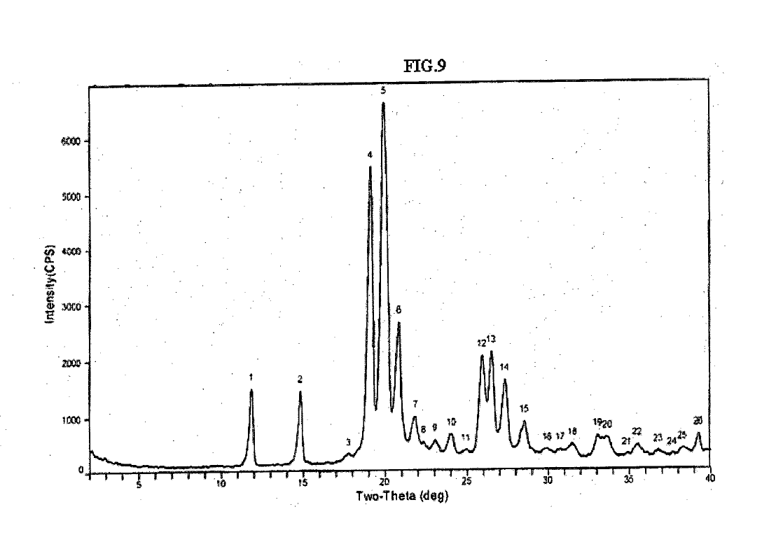

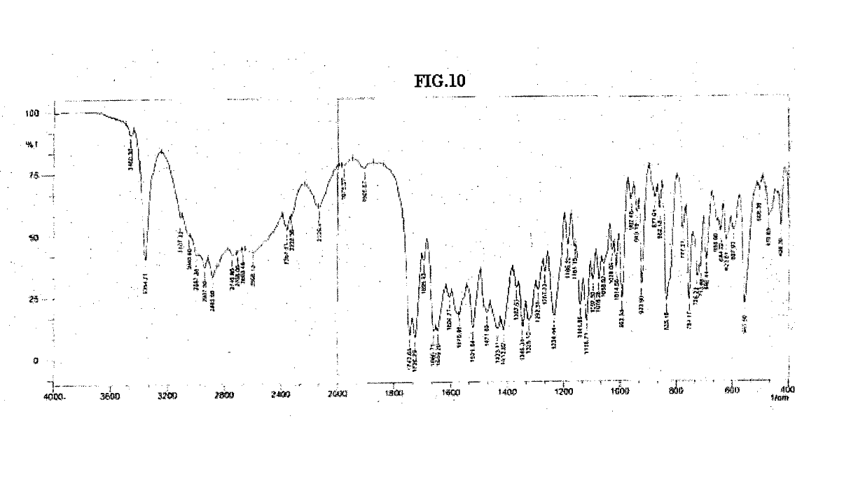

In another general aspect there is provided a solid state form of (S)-4-(4-(5-(aminomethyl)-2-oxo-oxazolidin-3-yl)phenyl)morpholin-3-one of Formula (J), which is characterized by XRD and IR as depicted in FIG. 9 and FIG. 10 respectively.

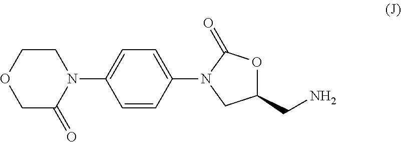 [SMILES: NC[C@H]1CN(c2ccc(N3CCOCC3=O)cc2)C(=O)O1]

A solid state form of (S)-4-(4-(5-(aminomethyl)-2-oxo-oxazolidin-3-yl)phenyl)morpholin-3-one of Formula (J) having characteristic peaks in XRD at about 11.8, 14.8, 19.1, 19.9, 20.8 and 26.4 and additional peaks at about 17.7, 21.8, 23.0, 24.0, 25.9, 27.3, 28.5 and 31.5±0.2 degrees two-theta.

A solid state form of (S)-4-(4-(5-(aminomethyl)-2-oxooxazolidin-3-yl)phenyl)morpholin-3-one of Formula (J) having IR absorption bands at about 3460, 3354, 3107, 2937, 2883, 2740, 2357, 2129, 1905, 1743, 1726, 1660, 1645 1521, 1433, 1413, 1346, 1325, 1234, 1141, 1118, 1076, 993, 923, 835, 754, 629, 555, 470 and 428±2 cm⁻¹.

In another general aspect there is provided an improved process for the preparation of 4-(4-aminophenyl)morpholin-3-one compound of Formula (C) and (S)-2-(oxiran-2-ylmethyl)isoindoline-1,3-dione of Formula (E) as shown below:

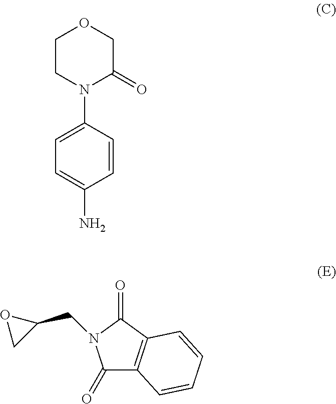 [SMILES: Nc1ccc(N2CCOCC2=O)cc1 ; O=C1c2ccccc2C(=O)N1C[C@H]1CO1]

In general aspect there is provided an improved process for the preparation of 4-(4-aminophenyl)morpholin-3-one of Formula (C),

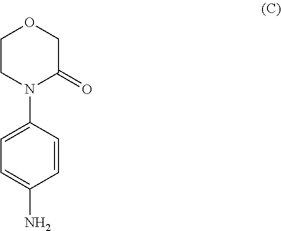 [SMILES: Nc1ccc(N2CCOCC2=O)cc1]

the process comprising hydrogenating 4-(4-nitrophenyl)morpholin-3-one of Formula (B) in the presence of a hydrogenation catalyst in a halogenated solvent to obtain the 4-(4-aminophenyl) morpholin-3-one compound of Formula (C). The hydrogenation is carried out in presence of catalyst selected from SnCl₂, Raney-Ni, Pd/C, Pt/C, and PtO₂. The halogenated solvent may be selected from methylenedichloride, ethylenedichloride, chlorobenzene, chloroform, carbon tetrachloride. In particular, hydrogenation of 4-(4-nitrophenyl)morpholin-3-one carried out in presence of Pd/C under reduced pressure in methylene dichloride.

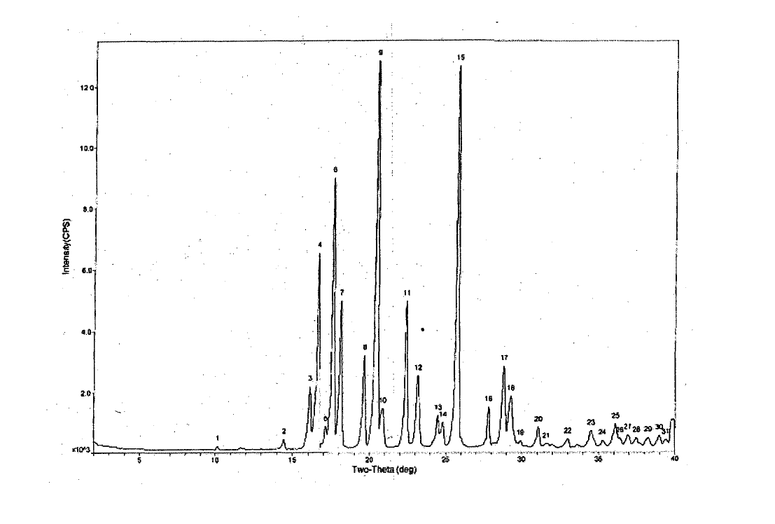

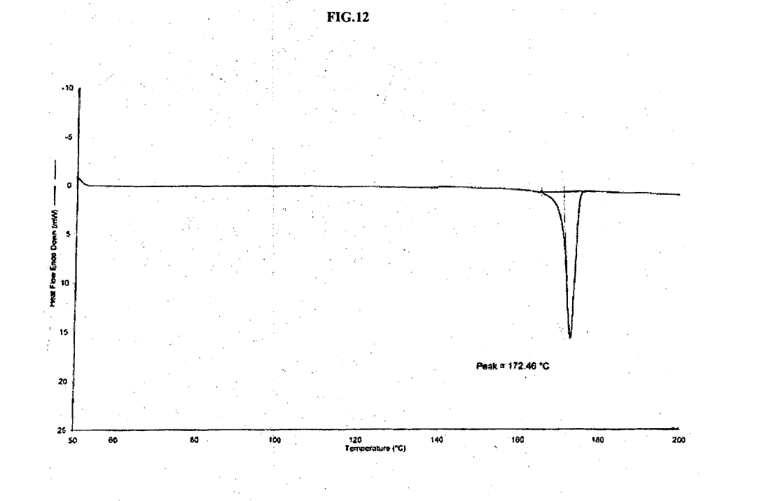

In another general aspect there is provided a solid state form of 4-(4-aminophenyl)morpholin-3-one of Formula (C), which is characterized by XRD and DSC as depicted in FIG. 11 and FIG. 12 respectively.

A solid state form of 4-(4-aminophenyl)morpholin-3-one of Formula (C) is characterized by X-ray powder diffraction having characteristic peaks at about 16.1, 16.6, 17.6, 18.1, 19.6, 20.4, 22.4, 23.1, 25.7, 28.8 and 29.2 and having additional peaks at about 10.1, 14.4, 17.1, 20.8, 24.4, 24.8, 27.8, 31.0, 32.9, 34.5 and 36.1±0.2 degree 2θ.

A solid state form of 4-(4-aminophenyl)morpholin-3-one of Formula (C) is characterized by Differential Scanning calorimetry (DSC) having peak at about 172° C.

In another general aspect there is provided an improved process for preparing a solid state form of 4-(4-aminophenyl)morpholin-3-one of Formula (C) as shown in below scheme (1)

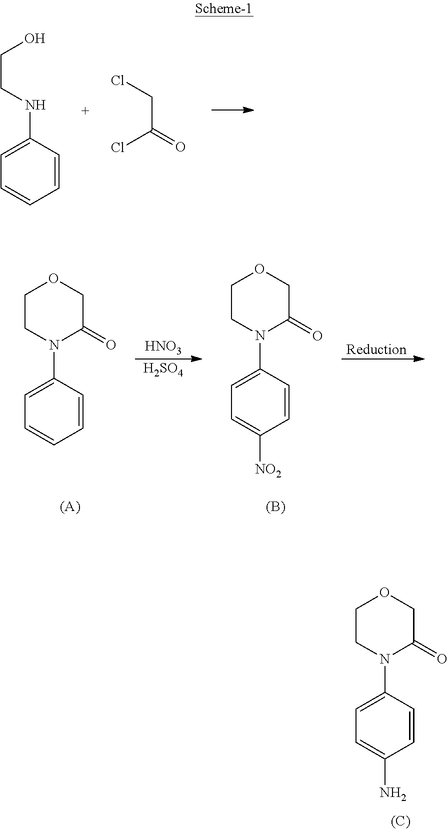

An improved process for the preparation of 4-(4-aminophenyl)morpholin-3-one of Formula (C), the process comprising:

i) reacting 2-(phenylamino)ethanol with 2-chloroacetyl chloride in the presence of a base in a suitable solvent to obtain 4-phenylmorpholin-3-one of Formula (A);
ii) reacting the 4-phenylmorpholin-3-one of Formula (A) with a nitrating agent to obtain 4-(4-nitro phenyl) morpholin-3-one of Formula (B); and
iii) hydrogenating the 4-(4-nitrophenyl)morpholin-3-one of formula (B) in the presence of a hydrogenation catalyst in a halogenated solvent to obtain the 4-(4-aminophenyl) morpholin-3-one compound of formula (C).

In another general aspect there is provided use of the solid state form of 4-(4-aminophenyl) morpholin-3-one compound of Formula (C) in the preparation of rivaroxaban of Formula (1).

In another general aspect there is provided an improved process for the preparation of (S)-2-(oxiran-2-ylmethyl)isoindoline-1,3-dione of Formula (E) as shown in below scheme (2):

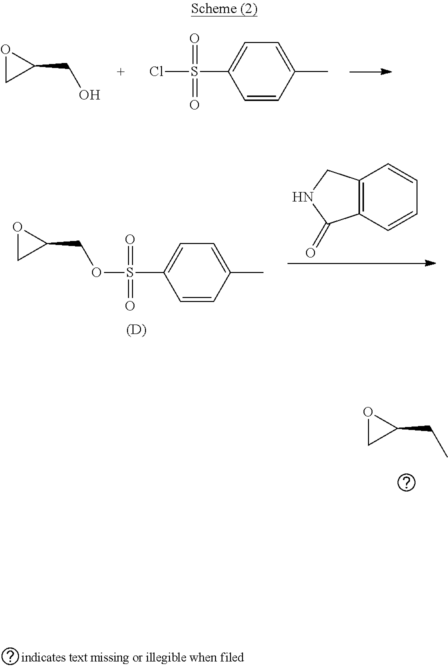

In another aspect there is provided an improved process for preparing (S)-2-(oxiran-2-ylmethyl) isoindoline-1,3-dione of Formula (E),

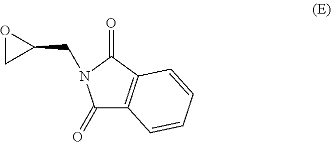 [SMILES: O=C1c2ccccc2C(=O)N1C[C@H]1CO1]

the process comprising:
i) reacting (S)-oxiran-2-yl methanol with p-toluene sulphonyl chloride in a suitable solvent in the presence of a base and a catalyst to obtain (R)-Glycidyl Tosylate (D); and
ii) reacting the (R)-Glycidyl Tosylate (D) with phthalamide in a suitable solvent in the presence of a base and a phase transfer catalyst to obtain (S)-2-(oxiran-2-ylmethyl) isoindoline-1,3-dione of Formula (E).

In particular, the suitable solvent used at step (i) may include one or more of C₁-C₅ alcohols, esters, ethers, halogenated solvent, aromatic hydrocarbons, water or a mixture thereof.

The base used at step (i) may include one or more of an alkali metal or alkaline metal hydroxides, carbonates and bicarbonate, and organic base such as triethylamine, diisoproypl ethylamine, diisopropylamine, preferably triethylamine.

The catalyst used at step (i) is selected from the group consisting of N,N-dimethylamino pyridine (DMAP), diisopropylamine (DIPA), diisopropyethylamine (DIPEA) etc; preferably DMAP.

The suitable solvent used at step (ii) is selected from one or more of C₁-C₅ esters, ketones, DMF, DMSO, halogenated solvents, aromatic hydrocarbons, water or a mixture thereof, preferably acetone.

The base used at step (ii) may be selected from one or more of alkali or alkaline earth metal hydroxides, alkoxides, carbonates or bicarbonates or an organic base. The base may be selected from one or more of sodium hydroxide, potassium hydroxide, sodium methoxide, potassium tert-butoxide, sodium carbonate, potassium carbonate, sodium bicarbonate, triethyl amine, diisopropyl amine, dimethyl amine, diisopropylethylamine, diisopropylmethyl amine, pyridine, piperidine, morpholine and N-methyl piperidine; preferably potassium carbonate.

The phase transfer catalyst used at step (ii) may be selected from one or more of TBAB, TBAC, TBAF, crown ethers etc; preferably TBAB.

In another general aspect there is provided a solid state form of acid addition salts of (S)-4-(4-(5-(aminomethyl)-2-oxo-oxazolidin-3-yl)phenyl) morpholin-3-one of Formula (J1),

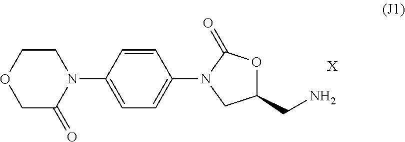

wherein X represents inorganic or organic acids, characterized by XRD and DSC.

The acid used for the formation of salt with the compound of formula (J) may be selected from inorganic acids or organic acids. Inorganic acids such as hydrochloric acid, hydrobromic acid, phosphoric acid or sulphuric acid. Organic acids such sulphonic acids, oxalic acid, formic acid, acetic acid, trifluoroacetic acid, propionic acid, maleic acid, succinic acid, fumaric acid, malic acid, citric acid, tartaric acid, lactic acid, benzoic acid, or methanesulphonic acid, mandelic acid, L(+)-mandelic acid, D(−)-mandelic acid, benzenesulphonic acid, toluenesulphonic acid or naphthalenedisulphonic acid.

The preferred acids are formic acid, oxalic acid, succinic acid and L(+)-mandelic acid.

In another general aspect, there is provided a solid state form of the acid addition salts of (S)-4-(4-(5-(aminomethyl)-2-oxo-oxazolidin-3-yl)phenyl) morpholin-3-one of Formula (J1)

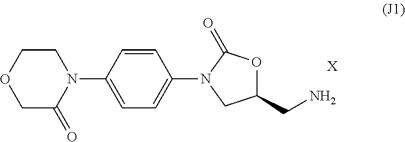

having purity greater than about 99%, specifically greater than about 99.8% as measured by HPLC.

In another general aspect, there is provided use of solid state form of the acid addition salt of (S)-4-(4-(5-(aminomethyl)-2-oxo-oxazolidin-3-yl)phenyl) morpholin-3-one of Formula (J1) in the preparation of rivaroxaban of Formula (1).

In another general aspect, there is provided a solid state form of the formate salt of (S)-4-(4-(5-(aminomethyl)-2-oxo-oxazolidin-3-yl)phenyl) morpholin-3-one of Formula (JF),

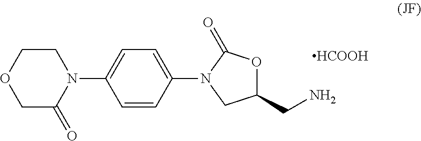 [SMILES: NC[C@H]1CN(c2ccc(N3CCOCC3=O)cc2)C(=O)O1 ; "·HCOOH"]

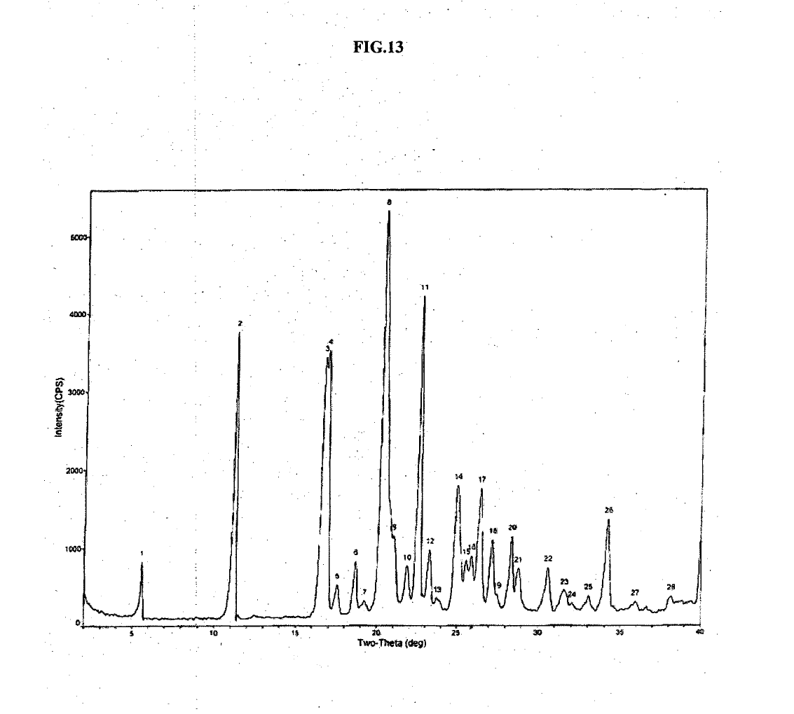

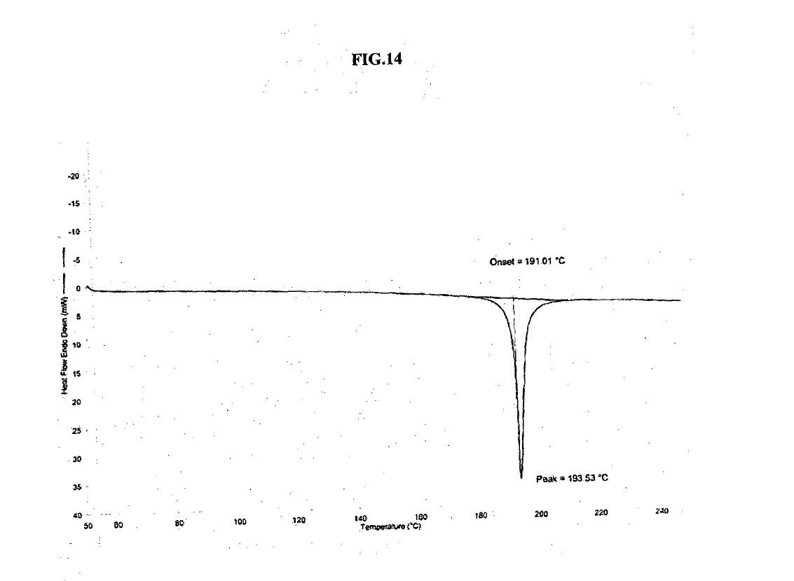

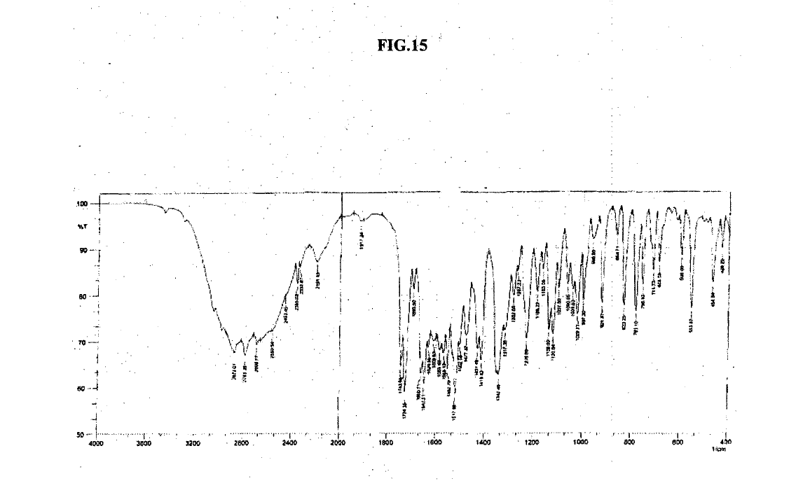

which is characterized by X-ray powder diffraction (XRD), DSC and IR as depicted in FIG. 13, FIG. 14 and FIG. 15 respectively.

A solid state form of the formate salt of (S)-4-(4-(5-(aminomethyl)-2-oxo-oxazolidin-3-yl)phenyl) morpholin-3-one of Formula (JF), which is characterized by XRD having peaks at about 5.5, 11.1, 16.6, 16.8, 18.6, 20.3, 22.5, 24.9, 26.4, 27.1, 28.3, 30.5, 34.1 and having additional peaks at about 17.5, 19.2, 20.9, 21.8, 23.4, 25.5, 25.9, 28.7, 31.5, 32.0, 33.0, 35.9 and 38.1±0.2 degree 20.

A solid state form of the formate salt of (S)-4-(4-(5-(aminomethyl)-2-oxo-oxazolidin-3-yl)phenyl) morpholin-3-one of Formula (JF), which is characterized by Differential Scanning calorimetry (DSC) having peak at about 193.5° C.

A solid state form of the formate salt of (S)-4-(4-(4-(5-(aminomethyl)-2-oxo-oxazolidin-3-yl)phenyl) morpholin-3-one of Formula (JF), which is characterized by Infrared (IR) having peaks at about 2872, 2783, 2688, 2355, 2191, 1917, 1743, 1724, 1693, 1660, 1647, 1552, 1517, 1477, 1431, 1413, 1342, 1282, 1228, 1186, 1138, 1120, 1097, 1060, 1022, 997, 960, 921, 864, 833, 783, 756, 711, 688, 596, 553, 464 and 428 cm⁻¹.

In another general aspect, there is provided a process for preparation of the solid state form of the formate salt of (S)-4-(4-(5-(aminomethyl)-2-oxo-oxazolidin-3-yl)phenyl) morpholin-3-one of Formula (JF), the process comprising:
(i) reacting (S)-2-((2-oxo-3-(4-(3-oxo-morpholino)phenyl)oxazolidin-5-yl)methyl) isoindoline-1,3-dione of Formula (I) with a base in a suitable solvent;
(ii) reacting with formic acid; and
(iii) isolating solid state form of the formate salt of (S)-4-(4-(5-(aminomethyl)-2-oxooxazolidin-3-yl) phenyl)morpholin-3-one (JF).

The suitable solvent for step (i) may be selected from one or more of hydrocarbons, nitriles, amides, alcohol, ketones, ester and the like. In particular, the suitable solvent may include toluene, xylene, ethylbenzene dimethyl formamide, dimethyl acetamide, acetonitrile, C₁-C₄ straight chain or branched alcohols, acetone, methyl isobutyl ketone, methyl ethyl ketone, ethyl acetate, isopropyl acetate, butyl acetate, halogenated solvent such as methylene dichloride; preferably methanol.

The base for step (i) may be one or more of hydrazine hydrate, C₁-C₅ amines; preferably 18% monomethyl amine in methanol.

In another general aspect, there is provided use of the solid state form of the formate salt of (S)-4-(4-(5-(aminomethyl)-2-oxo-oxazolidin-3-yl)phenyl) morpholin-3-one of Formula (JF) in the preparation of rivaroxaban of Formula (1).

In another general aspect, there is provided a solid state form of the oxalate salt of (S)-4-(4-(5-(aminomethyl)-2-oxo-oxazolidin-3-yl)phenyl) morpholin-3-one of Formula (JO),

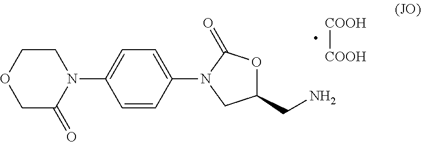

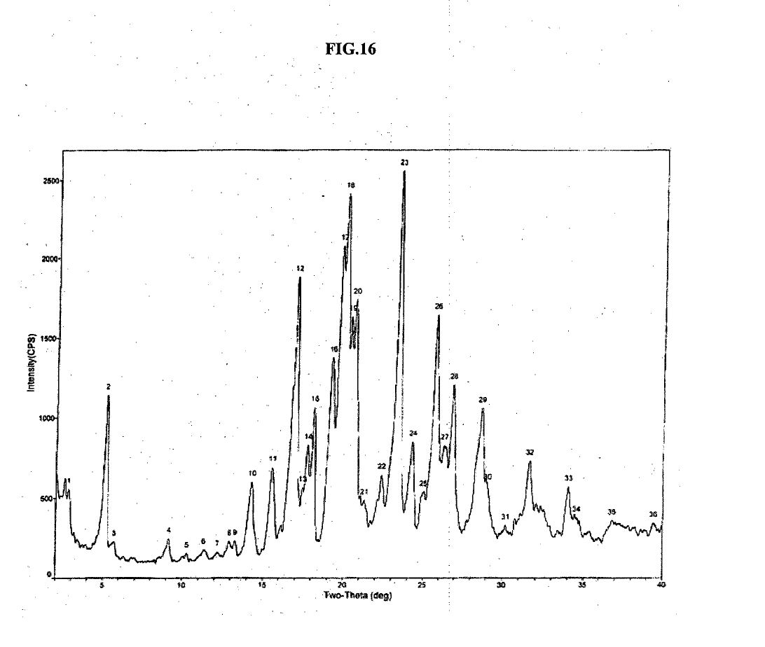

which is characterized by X-ray powder diffraction (XRD) as depicted in FIG. 16.

The solid state form of the oxalate salt of (S)-4-(4-(5-(aminomethyl)-2-oxo-oxazolidin-3-yl)phenyl) morpholin-3-one of Formula (JO) is characterized by XRD having peaks at about 2.8, 5.1, 9.1, 14.2, 15.5, 16.9, 17.7, 18.0, 19.1, 19.7, 20.3, 20.5, 23.3, 24.2, 25.7, 26.8, 28.6 and having additional peaks at about 5.6, 10.2, 11.3, 12.1, 12.9, 13.2, 17.4, 21.2, 22.3, 24.9, 26.2, 29.0, 30.1, 31.5 and 34.0±0.2 degree 2θ.

In another general aspect, there is provided use of the solid state form of the oxalate salt of (S)-4-(4-(5-(aminomethyl)-2-oxo-oxazolidin-3-yl)phenyl)morpholin-3-one of Formula (JO) in the preparation of rivaroxaban of Formula (1).

In another general aspect, there is provided a solid state form of the succinate salt of (S)-4-(4-(5-(aminomethyl)-2-oxo-oxazolidin-3-yl)phenyl) morpholin-3-one of Formula (JS),

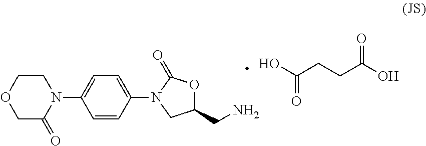 [SMILES: NC[C@H]1CN(c2ccc(N3CCOCC3=O)cc2)C(=O)O1 ; O=C(O)CCC(=O)O]

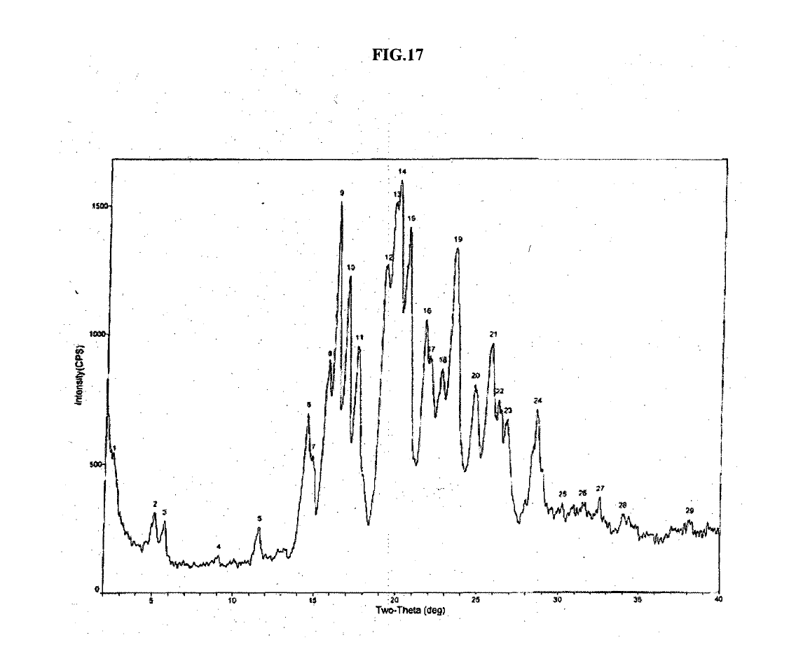

which is characterized by X-ray powder diffraction (XRD) as depicted in FIG. 17.

The solid state form of the succinate salt of (S)-4-(4-(5-(aminomethyl)-2-oxo-oxazolidin-3-yl)phenyl) morpholin-3-one of Formula (JS) is characterized by XRD having peaks at about 2.5, 5.1, 5.7, 11.5, 14.5, 15.7, 16.2, 16.8, 17.5, 19.1, 19.6, 19.8, 20.5, 21.5, 21.8, 23.4, 24.6, 25.7, 26.2, 26.7, 28.5 and having additional peaks at about 9.0, 14.8, 22.6, 30.1, 31.4, 32.5, and 33.9, ±0.2 degree 2θ.

In another general aspect, there is provided use of the solid state form of the succinate salt of (S)-4-(4-(5-(aminomethyl)-2-oxo-oxazolidin-3-yl)phenyl) morpholin-3-one of Formula (JS) in the preparation of rivaroxaban of Formula (1).

In another general aspect, there is provided a solid state form of the mandelate salt of (S)-4-(4-(5-(aminomethyl)-2-oxo-oxazolidin-3-yl)phenyl)morpholin-3-one of Formula (JM)

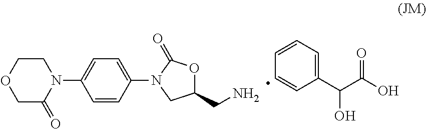 [SMILES: NC[C@H]1CN(c2ccc(N3CCOCC3=O)cc2)C(=O)O1 ; O=C(O)C(O)c1ccccc1]

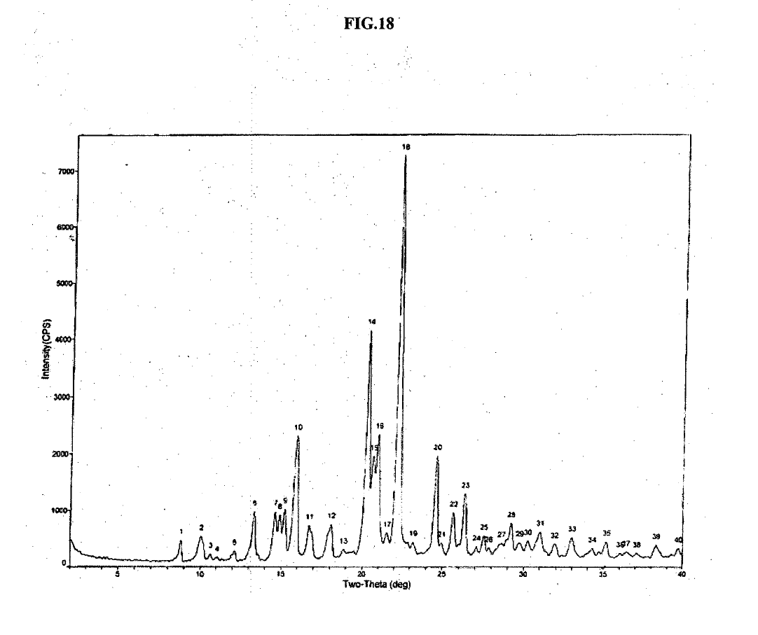

which is characterized by X-ray powder diffraction (XRD) as depicted in FIG. 18.

The solid state form of the mandelate salt of (S)-4-(4-(5-(aminomethyl)-2-oxo-oxazolidin-3-yl)phenyl)morpholin-3-one of Formula (JM) is characterized by XRD having peaks at about 13.3, 14.6, 15.2, 15.9, 20.2, 20.9, 22.2, 24.6, 25.6, 26.3 and having additional peaks at about 8.8, 10.0, 12.1, 16.7, 18.0, 20.6, 27.5, 29.2, 31.0, and 32.9±0.2 degree 2θ.

In another general aspect, there is provided use of the solid state form of the mandelate salt of (S)-4-(4-(5-(aminomethyl)-2-oxo-oxazolidin-3-yl)phenyl) morpholin-3-one of Formula (JM) in the preparation of rivaroxaban of Formula (1).

In another general aspect, there is provided an improved process for the preparation of rivaroxaban of Formula (1) or its pharmaceutical acceptable salts, solvates, and hydrates thereof,

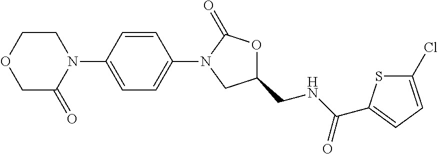 [SMILES: O=C(NC[C@H]1CN(c2ccc(N3CCOCC3=O)cc2)C(=O)O1)c1ccc(Cl)s1]

the process comprising:
i) reacting 4-(4-aminophenyl)morpholin-3-one of Formula (C) with (S)-2-(oxiran-2-ylmethyl)isoindoline-1,3-dione of Formula (E) in a suitable solvent in the absence of a base to obtain (R)-2-(2-hydroxy-3-(4-(3-oxo-morpholino)phenylamino)propyl)isoindoline-1,3-dione of Formula (F);
ii) reacting the (R)-2-(2-hydroxy-3-(4-(3-oxo-morpholino)phenylamino)propyl) isoindoline-1,3-dione of Formula (F) with a cyclizing agent in a suitable solvent in the presence of a catalyst to obtain (S)-2-((2-oxo-3-(4-(3oxo-morpholino)phenyl) oxazolidin-5-yl)methyl)isoindoline-1,3-dione of Formula (I);
iii) reacting the (S)-2-((2-oxo-3-(4-(3-oxo-morpholino)phenyl)oxazolidin-5-yl)methyl) isoindoline-1,3-dione of Formula (G) in presence of base in a suitable solvent to obtain (S)-4-(4-(5-(amino methyl)-2-oxooxazolidin-3-yl)phenyl)morpholin-3-one (J1); and
iv) reacting the (S)-4-(4-(5-(aminomethyl)-2-oxooxazolidin-3-yl)phenyl)morpholin-3-one of Formula (J) with 5-chlorothiophene-2-carboxylic acid chloride in a biphasic solvent system in the presence of a base to obtain rivaroxaban.

The suitable solvent at step (i) may include one or more of C₁-C₅ alcohols, esters, ethers, nitriles, tetrahydrofuran (THF), water, halogenated solvents, dimethylformamide, dimethyl sulfoxide, sulfolane, or a mixture thereof, preferably isopropanol.

The suitable solvent at step (ii) may include one or more of C₁-C₅ alcohols, esters, ethers, nitriles, tetrahydrofuran (THF), water, halogenated solvents, dimethylformamide, dimethyl sulfoxide, sulfolane, or a mixture thereof, preferably methylene dichloride (MDC).

The cyclizing agent at step (ii) comprises one or both of N,N-carbonyldiimidazole (CDI), and phosgene; preferably CDI.

The catalyst at step (ii) comprises one or more of N,N-dimethylaminopyridine (DMAP), diisopropylamine (DIPA), and diisopropyethylamine (DIPEA); preferably DMAP.

The suitable solvent at step (iii) may include one or more of hydrocarbons, nitriles, amides, alcohol, ketones, ester and the like. In particular, the suitable solvent comprises toluene, xylene, ethylbenzene dimethyl formamide, dimethyl acetamide, acetonitrile, C₁-C₄ straight chain or branched alcohols, acetone, methyl isobutyl ketone, methyl ethyl ketone, ethyl acetate, isopropyl acetate, butyl acetate, halogenated solvent such as methylene dichloride; preferably methanol.

The base at step (iii) is selected from group consisting of hydrazine hydrate, C₁-C₅ amines; preferably 18% monomethyl amine in methanol.

The suitable solvent for step (iv) in a biphasic solvent system is selected from the group of solvents such as hydrocarbons, nitriles, amides, alcohol, ketones, halogenated solvent, ester, toluene, xylene, ethylbenzene dimethyl formamide, dimethyl acetamide, acetonitrile, C₁-C₄ straight chain or branched alcohols, acetone; methyl isobutyl ketone, methyl ethyl ketone, ethyl acetate, isopropyl acetate, butyl acetate, preferably biphasic system of methylene dichloride (MDC)-water or acetonitrile-water.

The base at step (iv) comprises of an organic base or inorganic base. The organic base may include one or more of diisopropylethylamine, diisopropylamine, triethylamine, diethylamine, pyridine, N-methyl piperidine, piperidine, morpholine, pyridine, DBU, DABCO and the like. The inorganic base may include one or more of an alkali or an alkaline metal hydroxides, alkoxides, carbonates, and bicarbonate; particularly sodium hydroxide, potassium hydroxide, lithium hydroxide, sodium carbonate, potassium carbonate, cesium carbonate, sodium methoxide, potassium tert-butoxide. Particularly, the base may be sodium carbonate.

In another general aspect there is provided chirally pure (S)-2-((2-oxo-3-(4-(3-oxo-morpholino)phenyl)oxazolidin-5-yl)methyl)isoindoline-1,3-dione of Formula (I)

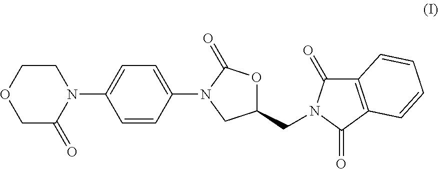 [SMILES: O=C1c2ccccc2C(=O)N1C[C@H]1CN(c2ccc(N3CCOCC3=O)cc2)C(=O)O1]

having chiral purity greater than about 99%, specifically greater than about 99.5%, more specifically greater than about 99.9% and most specifically greater than about 99.98% as measured by HPLC.

In another general aspect, there is provided use of chirally pure (S)-2-((2-oxo-3-(4-(3-oxo-morpholino)phenyl)oxazolidin-5-yl)methyl)isoindoline-1,3-dione of Formula (I) in the preparation of rivaroxaban of Formula (1).

In another general aspect, there is provided a process for the enrichment of chiral purity of the (S)-2-((2-oxo-3-(4-(3-oxo-morpholino)phenyl)oxazolidin-5-yl)methyl)isoindoline-1,3-dione of Formula (I)

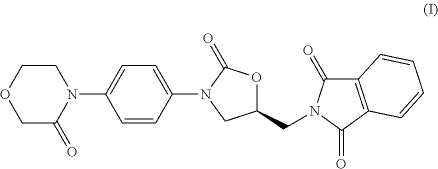 [SMILES: O=C1c2ccccc2C(=O)N1C[C@H]1CN(c2ccc(N3CCOCC3=O)cc2)C(=O)O1]

the process comprising:
i) crystallizing (S)-2-((2-oxo-3-(4-(3-oxo-morpholino)phenyl)oxazolidin-5-yl)methyl)isoindoline-1,3-dione of Formula (I) form, an organic solvent; and
ii) isolating the chirally pure (S)-2-((2-oxo-3-(4-(3-oxo-morpholino)phenyl)oxazolidin-5-yl)methyl)isoindoline-1,3-dione of Formula (I).

The organic solvent may include one or more of hydrocarbons, nitriles, amides, alcohol, ketones, halogenated solvent, ester selected from toluene, xylene, ethylbenzene dimethyl formamide, dimethyl acetamide, acetonitrile, C₁-C₄ straight chain or branched alcohols, acetone, methyl isobutyl ketone, methyl ethyl ketone, ethyl acetate, isopropyl acetate, butyl acetate or mixture thereof, preferably mixture of DMF-methanol.

In another general aspect, there is provided rivaroxaban of Formula (1) having a chiral purity of greater than about 99%, specifically greater than about 99.5%, more specifically greater than about 99.9%, and most specifically greater than about 99.98%.

In another general aspect, there is provided rivaroxaban of Formula (1) having a total purity of greater than about 99%, specifically greater than about 99.5%, more specifically greater than about 99.9%, and most specifically greater than about 99.98% as measured by HPLC.

In another general aspect, there is provided a particle size of rivaroxaban of Formula (1).

In another general aspect there is provided rivaroxaban having an average particle size in the range of 5 to 300 microns, preferably 20 to 150 microns, more preferably 50 to 400 microns. The term “average particle size” or “particle size” as used herein refers to the volume mean diameter of particles.

In another general aspect there is provided rivaroxaban having particle size in terms of d₉₀ less than about 100 microns.

In another general aspect there is provided rivaroxaban having particle size in terms of d₉₀ less than about 50 microns.

In another general aspect there is provided rivaroxaban having particle size in terms of d₉₀ less than about 10 microns.

‘Pharmaceutically acceptable salts’ as used herein can preferably be salts of rivaroxaban with an inorganic acid or an organic acids. The inorganic acid comprises hydrochloric acid, hydrobromic acid, phosphoric acid or sulphuric acid. The organic acid comprises organic carboxylic or sulphonic acids, such as, for example oxalic acid, acetic acid, formic acid, succinic acid, trifluoroacetic acid, propionic acid, maleic acid, fumaric acid, malic acid, citric acid, tartaric acid, lactic acid, benzoic acid, or methanesulphonic acid, ethanesulphonic acid, benzenesulphonic acid, toluenesulphonic acid or naphthalenedisulphonic acid. Other pharmaceutically acceptable salts are without limitation, salts with customary bases, such as for example, alkali metal salts such as sodium or potassium salts, alkaline earth metal salts such as calcium or magnesium salts or ammonium salts, derived from ammonia or organic amines, such as, for example, diethylamine, triethylamine, ethyldiisopropylamine, procaine, dibenzylamine, N-methylmorpholine or methylpiperidine.

Rivaroxaban of Formula (1) according to the present invention is stable and is particularly suitable for preparing medicaments.

In general aspect there is provided a pharmaceutical composition of rivaroxaban having particle size in terms of d₉₀ less than about 100 micron, preferably, less than about 50 micron, more preferably less than about 10 micron.

The pharmaceutical compositions may be in a solid or liquid dosage form. Exemplary solid dosage forms include tablets, capsules, sachets, lozenges, powders, pills, pellets, or granules. The solid dosage form may be, for example, a immediate release dosage form, a fast melt dosage form, orally disintegrating dosage form, modified release dosage form, lyophilized dosage form, delayed release dosage form, extended release dosage form, prolonged release dosage form, pulsatile dosage form, mixed immediate and modified release dosage form, or a combination thereof. Solid dosage forms are preferred. More preferably, the solid dosage form is an immediate release dosage form offering advantages regarding the bioavailability of the active compound.

Pharmaceutical dosage forms comprising rivaroxaban can be prepared by a process comprising the steps of mixing rivaroxaban according to the present invention with at least one pharmaceutically acceptable excipient and forming the mixture into a pharmaceutical dosage form. Rivaroxaban and the one or more excipients can be mixed in the presence or in the absence of solvent.

Preferably, the process for preparation of rivaroxaban compound of Formula (1) as per the present invention is shown in below scheme-3 and scheme-4:

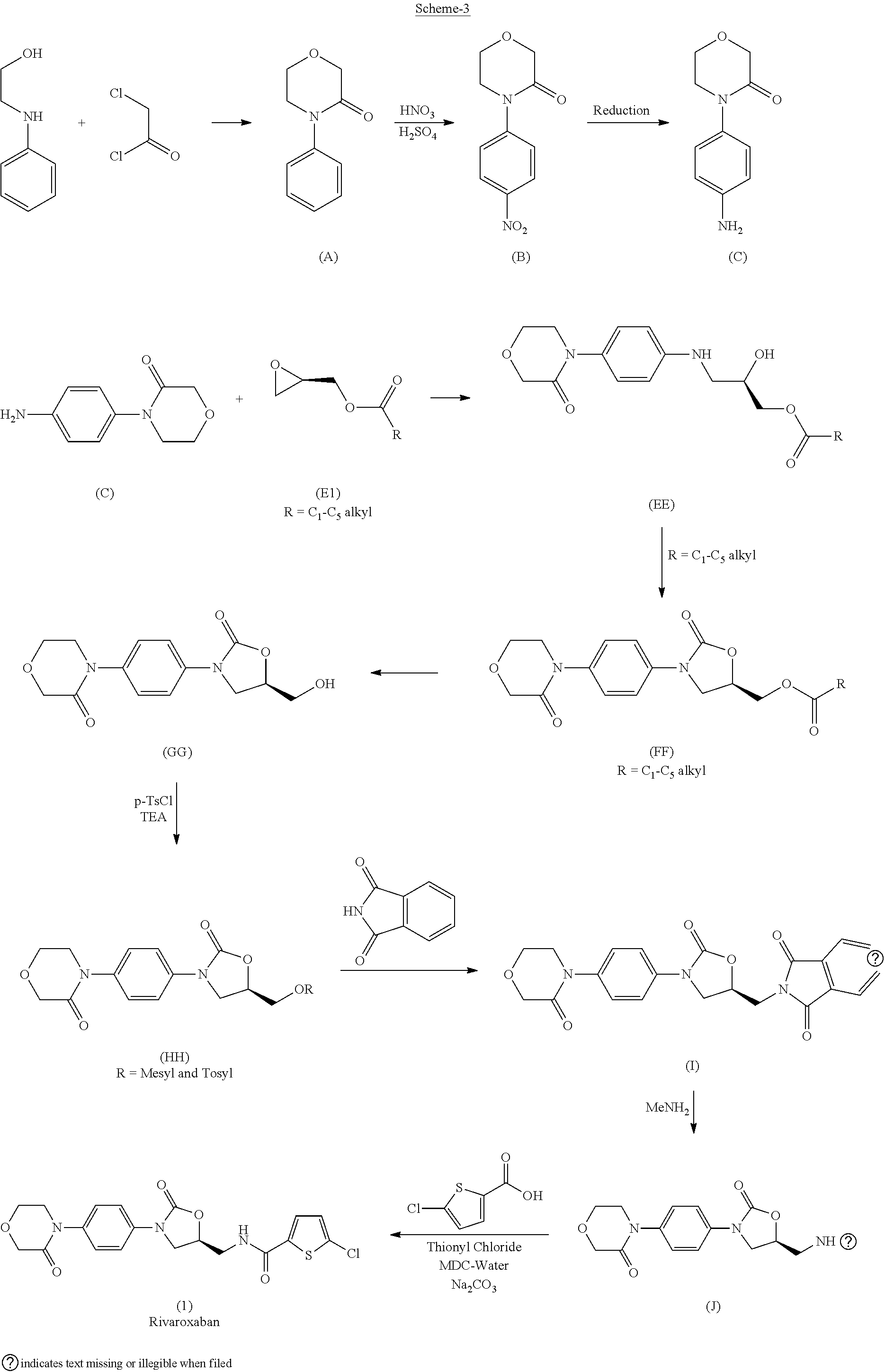

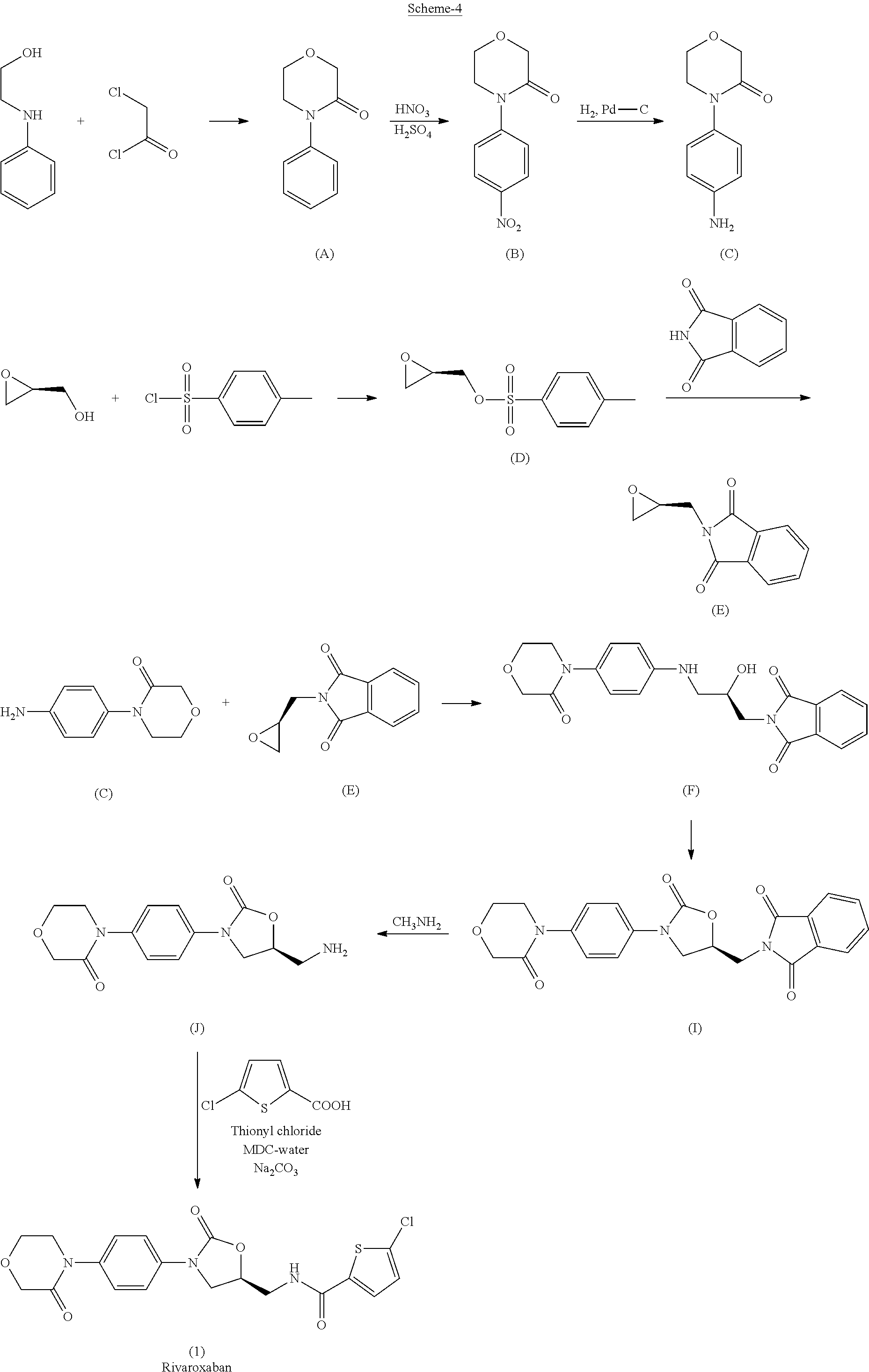

The present invention is further illustrated by the following examples which are provided merely to be exemplary of the invention and do not limit the scope of the invention. Certain modification and equivalents will be apparent to those skilled in the art and are intended to be included within the scope of the present invention.

### Example-1

### Preparation of 4-Phenylmorpholin-3-one of Formula (A)

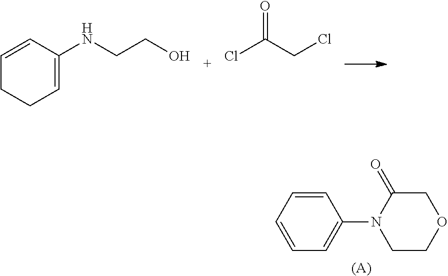

100 ml IPA and 100 g 2-phenylaminoethanol were added to RBF at 25° C. to 35° C. followed by stirring for 10 min. To the reaction mass 100 ml water added and temperature raised to 35° C. to 40° C. Further NaOH solution (180.78 g NaOH+350 mL water) was added in pressure equalizing funnel. Similarly 246.98 g chloroacetyl chloride was added in another pressure equalizing dropping funnel. The NaOH solution and chloroacetyl chloride were added simultaneously at such a rate that pH maintained between 12.5-13.0. The reaction mass was stirred for 60 min. 35° C. to 40° C. water to afford title compound as 4-Phenylmorpholin-3-one (A)

### Example-2

### Preparation of 4-(4-Nitrophenyl)morpholin-3-one of Formula (B)

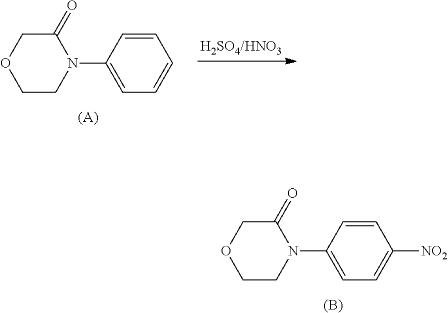

150 ml Conc. Sulfuric acid and 100 gm 4-phenyl morpholine-3-one of Formula (A) were added to RBF at 25° C. to 35° C. followed by cooling to 0° C. to 5° C. and stirring for 15 min. Further, 56.92 gm Nitric Acid (strength 70%) was added over a period of 90 min. and stirred for 60 min. at 0° C. to 5° C. To another RBF 1.5 L process water was added at 25° C. to 35° C. and cooled to 0° C. to 5° C. The reaction mass was added to RBF containing water at 0° C. to 5° C. and stirred for 60 min. The product was filtered and washed with 2×100 ml water to afford the title compound as 4-(4-Nitrophenyl)morpholin-3-one (B).

### Example-3

### Preparation of 4-(4-Aminophenyl)morpholin-3-one of Formula (C)

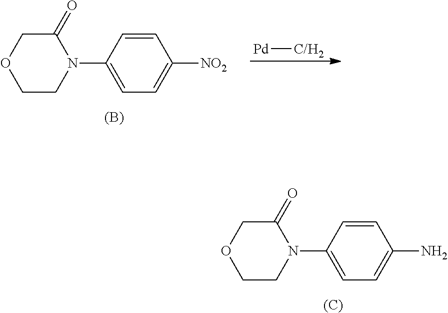

1.7 L MDC, 100 g 4-(4-nitrophenyl)morpholin-3-one of Formula (B) and 5 g Pd—C were added to 5 L Autoclave The assembly was flushed two times with N₂ gas at 5 Kg pressure and with H₂ gas at 5 Kg pressure at 25° C. to 35° C. The pressure of H₂ gas was set at 5 Kg and temperature raised to 75° C. to 80° C. The reaction mass was cooled to 25° C. to 35° C. and H₂ gas was released and the assembly was flushed with N₂ gas at 5 Kg pressure. Pd—C was filtered off through celite and washed 500 ml MDC. Excess of solvent distilled out under reduced pressure below 45° C. and further cooled to 25° C. to 35° C. The product was washed filtered and washed with 100 ml mixture of MDC-hexane to afford the title compound as 4-(4-aminophenyl)morpholin-3-one.

### Example-4

### Preparation of (R)-2-Hydroxy-3-(4-(3-oxo-morpholino)phenylamino)propyl butyrate of Formula (E′)

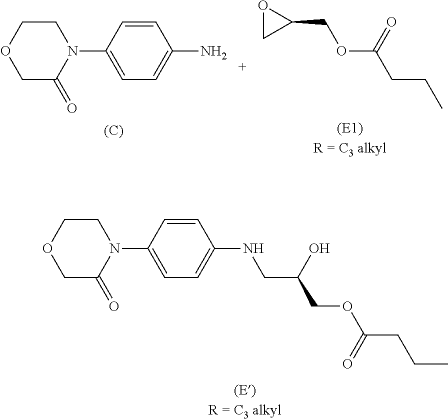

100 g 4-(4-Aminophenyl)morpholin-3-one of Formula (C) and ethanol (1000 ml) were added to RBF at 25° C. to 35° C. and heated to 75° C. to 80° C. followed by addition of 74.26 g (R)-Glycidyl butyrate. The reaction mass was stirred for 12 hrs. Ethanol was completely under reduced pressure at 55° C. to 60° C. Further, 500 ml water was added at 25° C. to 35° C. and stirred for 60 min. The reaction mass was cooled to 20° C. to 30° C. The product was filtered and washed with 500 ml water to afford the title compound as (R)-2-Hydroxy-3-(4-(3-oxo-morpholino)phenylamino)propyl butyrate (E′).

### Example-5

### Preparation of (R)-(2-oxo-3-(4-(3-oxo-morpholino)phenyl)oxazolidin-5-yl)methyl butyrate of Formula (F′)

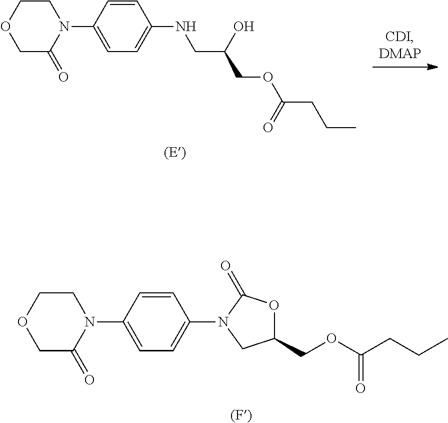 [SMILES: CCCC(=O)OC[C@H](O)CNc1ccc(N2CCOCC2=O)cc1 ; CCCC(=O)OC[C@H]1CN(c2ccc(N3CCOCC3=O)cc2)C(=O)O1]

100 g (R)-2-Hydroxy-3-(4-(3-oxo-morpholino)phenylamino)propyl butyrate of Formula (E′) and MDC (1000 ml) were added to RBF at 25° C. to 35° C. and heated to 40° C. TO 45° C. Further, 72 g N,N-Carbonyl diimidazole (CDI) and 0.5 g DMAP were added and stirred for 3-4 hours. The reaction mass was cooled to 25° C. to 35° C. followed by addition of dilute HCl solution and stirred for 30 min. MDC layer was separated and excess of MDC was evaporated under reduced pressure to afford title compound as (R)-(2-oxo-3-(4-(3-oxo-morpholino)phenyl)oxazolidin-5-yl)methyl butyrate of Formula (F′).

### Example-6

### Preparation of (R)-4-(4-(5-(hydroxymethyl)-2-oxo-oxazolidin-3-yl)phenyl)morpholin-3-one

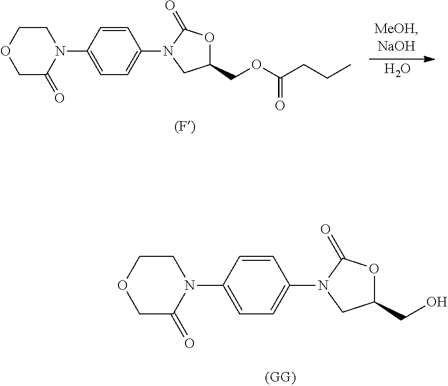 [SMILES: CCCC(=O)OC[C@H]1CN(c2ccc(N3CCOCC3=O)cc2)C(=O)O1 ; O ; O=C1COCCN1c1ccc(N2C[C@H](CO)OC2=O)cc1]

100 g (R)-(2-oxo-3-(4-(3-oxo-morpholino)phenyl)oxazolidin-5-yl)methyl butyrate of Formula (F′) and 500 ml Methanol were added to RBF at 25° C. to 35° C. followed by addition of 20 ml water and 13 g NaOH. The reaction was heated to 60° C. to 65° C. for 1 hour. Further, solvent was evaporated completely followed by addition of 300 mL MDC and stirring for 30 min. and product was filtered and solvent was evaporated to afford the title compound as (R)-4-(4-(5-(hydroxymethyl)-2-oxo-oxazolidin-3-yl)phenyl)morpholin-3-one of Formula (GG).

### Example-7

### Preparation of (R)-(2-oxo-3-(4-(3-oxo-morpholino)phenyl)oxazolidin-5-yl)methyl 4-methylbenzene sulfonate of Formula (H)

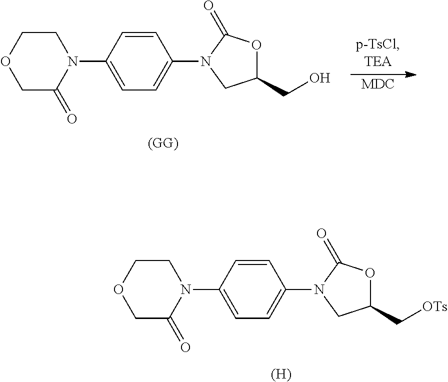

100.0 g (R)-4-(4-(5-(hydroxymethyl)-2-oxo-oxazolidin-3-yl)phenyl)morpholin-3-one of Formula (GG) and 250 ml MDC were added to RBF at 25° C. to 35° C. followed by addition of 52.85 gm TEA and 83.14 gm p-Toluenesulfonyl chloride. The reaction mass was stirred for 24 hours at 25° C. to 35° C. Further, solvent warms evaporated completely and followed by addition of 100 mL brine solution water and 300 mL MDC. The reaction mass was stirred for 30 min. and followed by evaporation of solvent to afford title compound as (R)-(2-oxo-3-(4-(3-oxo-morpholino)phenyl)oxazolidin-5-yl)methyl 4-methylbenzene sulfonate of Formula (H).

### Example-8

### Preparation of (S)-2-((2-oxo-3-(4-(3-oxomorpholino)phenyl)oxazolidin-5-yl)methyl)isoindoline-1,3-dione of Formula (I)

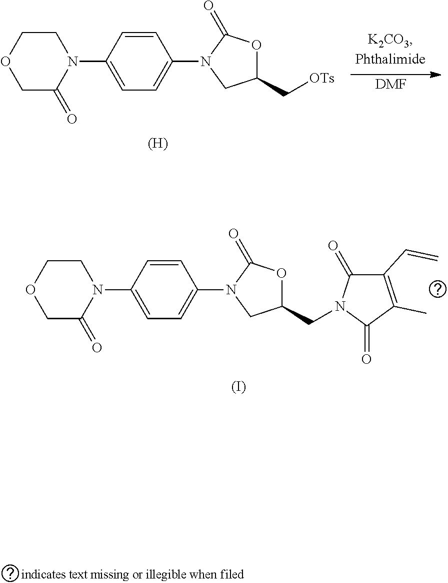

500 mL DMF and 30 g Phthalimide, 91.08 g K₂CO₃ and 100 g (R)-(2-oxo-3-(4-(3-oxo-morpholino) phenyl)oxazolidin-5-yl)methyl 4-methylbenzenesulfonate of Formula (H) were added to RBF at 25° C. to 35° C. The reaction mass was heated to 40° C. to 45° C. and stirred for 10 hours. Further, reaction mass was treated 1000 ml 10% HCl solution and 500 ml MDC. The MDC layer was washed with 3×500 mL 10% HCL solution to afford the title compound as (S)-2-((2-oxo-3-(4-(3-oxo-morpholino)phenyl)oxazolidin-5-yl)methyl)isoindoline-1,3-dione of Formula (I).

### Example-9

### Preparation of formate salt of (S)-4-(4-(5-(Aminomethyl)-2-oxooxazolidin-3-yl)phenyl) morpholin-3-one of Formula (JF)

100 g (S)-2-((2-oxo-3-(4-(3-oxo-morpholino)phenyl)oxazolidin-5-yl)methyl)isoindoline-1,3-dione of Formula (I), 1000 ml Methanol and 119 g methyl amine were added to RBF at 25° C. to 35° C. The reaction was heated to 60° C. to 65° C. for 1-2 hours. To the reaction mass formic acid (15 g) was added till to pH 5.5 to 6.0 and maintained for 30 minutes. The reaction mass was cooled to 45° to 50° C. and maintained for 30 minutes. The reaction mass was cooled to 25° to 35° C. and maintained for 30 minutes. The product was filtered and washed with methanol (50 ml×2) afforded the formate salt of (S)-4-(4-(5-(Aminomethyl)-2-oxooxazolidin-3-yl)phenyl)morpholin-3-one of Formula (JF).

### Example-10

### Preparation of Rivaroxaban

Thionyl chloride (10 ml) and 5-chlorothiophene-2-carboxylic acid (5 g) were added at 25° C. to 35° C. and heated to 65° C. to 70° C. and maintained for 60 minutes. Toluene (25 ml) was added and heated to 110° C. to 120° C. and excess thionyl chloride and toluene was distilled out. In another RBF formate salt of (S)-4-(4-(5-(aminomethyl)-2-oxo-oxazolidin-3-yl)phenyl)morpholin-3-one of Formula (JF) (10 g) was added and water (50 ml) and MDC (100 ml) was added at 25° C. to 35° C. and cooled to 0° C. to 5° C. followed by addition of sodium carbonate (4.5 g). To the reaction mass above prepared acid chloride solution was added at 0° C. to 5° C. and raised to 25° C. to 35° C. The reaction mass was filtered and washed with water (20 ml). The wet cake was treated with MDC (50 ml) and stirred for 15 minutes. To the reaction mass methanol (50 ml) was added and stirred for 30 minutes and washed with mixture of MDC-methanol (1:1) (10 ml). The wet cake was treated with 50% HCl solution (100 ml) at 50° C. to 60° C. and stirred for 30 minutes. The reaction mass was filtered and washed with water (20 ml) afforded as crude rivaroxaban of Formula (1).

10 g of crude Rivaroxaban was treated with methanol (100 ml) at 50° C. to 55° C. and stirred for 30 minutes. The product was filtered and washed with methanol (10 ml) afforded the title compound as pure rivaroxaban. (free of MDC as residual solvent and HPLC purity: 99.98%)

### Example-11

### Preparation of (R)-oxiran-2-ylmethyl 4-methylbenzenesulfonate of Formula (D)

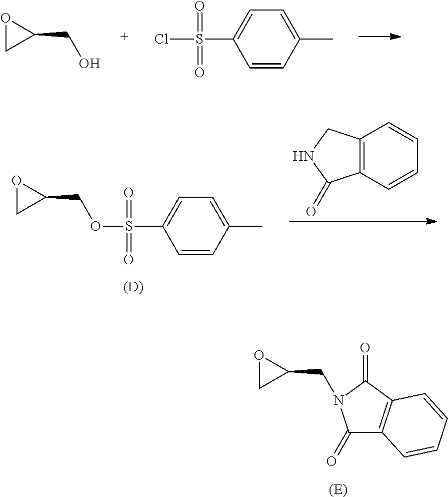

1000 ml MDC, 100.0 g (S)-Glycidol and 0.164 g triethylamine were added to RBF at 25° C. to 35° C. followed by addition of 255 g p-toluene sulphonyl chloride portion wise and 1.6 g DMAP. The reaction mass was stirred for 3-4 hours and product was filtered and washed with 200 ml MDC and further stirred for 1-2 hours. MDC layer was washed with dilute HCl solution and further with 2000 ml water, followed by separation of layers and evaporation of solvent to afford (R)-oxiran-2-ylmethyl 4-methylbenzenesulfonate of Formula (D).

### Example-12

### Preparation of (S)-2-(oxiran-2-ylmethyl)isoindoline-1,3-dione of Formula (E)

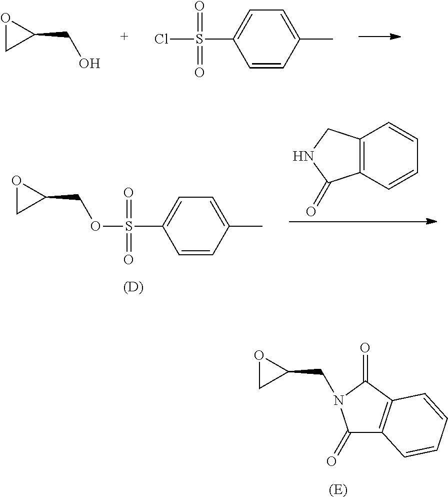

2 L Acetone, 64 g Phthalamide and 190 g K₂CO₃ were added to RBF at 25° C. to 35° C. and heated to 55° C. to 60° C. followed by addition of 100 g (R)-Glycidyl Tosylate of Formula (D) and 14 g TBAB and stirred for 12 hours at 55° C. to 60° C. The product was washed with 100 ml acetone followed by addition of 1500 ml water into the above mass and stirring for 2 hours to afford the title compound as (S)-2-(oxiran-2-ylmethyl)isoindoline-1,3-dione of Formula (E).

### Example-13

### (R)-2-(2-hydroxy-3-(4-(3-oxomorpholino)phenylamino)propyl)isoindoline-1,3-dione of Formula (F)

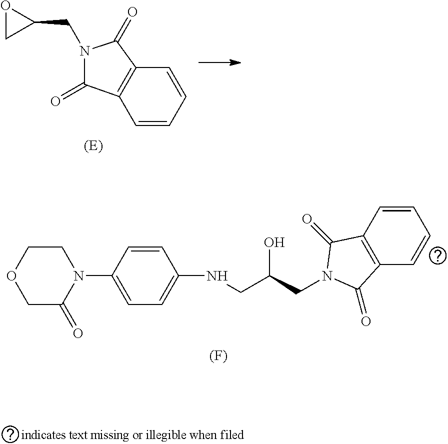 [SMILES: O=C1c2ccccc2C(=O)N1C[C@H]1CO1 ; O=C1c2ccccc2C(=O)N1C[C@H](O)CNc1ccc(N2CCOCC2=O)cc1 ; "(F)"]

100 g 4-(4-Aminophenyl)morpholin-3-one and ethanol (1000 ml) were added to RBF at 25° C. to 35° C. and heated to 75° C. to 80° C. followed by addition of 105 g (S)-2-(Oxiran-2-ylmethyl)isoindoline-1,3-dione of Formula (E). The reaction mass was stirred for 18 hrs. The reaction mass was cooled to 25° C. to 35° C. and stirred for 1 hour. The product was filtered and washed with 2×200 ml ethanol to afford the title compound as (R)-2-(2-hydroxy-3-(4-(3-oxomorpholino)phenylamino) propyl) isoindoline-1,3-dione of Formula (F)

### Example-14

### Preparation of (S)-2-((2-oxo-3-(4-(3-oxo-morpholino)phenyl)oxazolidin-5-yl)methyl)isoindoline-1,3-dione of Formula (I)

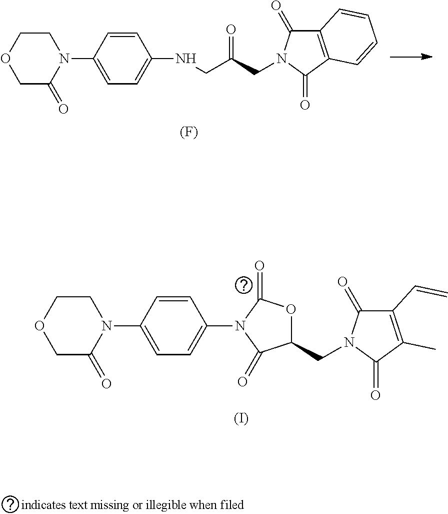

100 g (R)-2-(2-hydroxy-3-(4-(3-oxo-morpholino) phenyl amino) propyl) isoindoline-1,3-dione of Formula (F) and MDC (1000 ml) were added to RBF at 25° C. to 35° C. and heated to 40° C. to 45° C. Further, 61 g N,N-Carbonyl diimidazole (CDI) and 0.5 g DMAP were added and stirred for 3-4 hours. Excess MDC was evaporated under, reduced pressure. The reaction mass was cooled to 25° C. to 35° C. followed by addition of dilute HCl solution and stirred for 1 hour and finally filtered and washed with 2×200 ml water to afford title compound as (S)-2-((2-oxo-3-(4-(3-oxo-morpholino)phenyl)oxazolidin-5-yl)methyl)isoindoline-1,3-dione of Formula (I).

### Example-15

### Preparation of formate salt of (S)-4-(4-(5-(aminomethyl)-2-oxo-oxazolidin-3-yl)phenyl)morpholin-3-one of Formula (JF)

100 g (S)-2-((2-oxo-3-(4-(3-oxo-morpholino)phenyl)oxazolidin-5-yl)methyl)isoindoline-1,3-dione of Formula (I), 1000 ml methanol and 119 g methyl amine were added to RBF at 25° C. to 35° C. The reaction was heated to 60° C. to 65° C. for 1-2 hours. To the reaction mass formic acid (15 g) was added till to pH 5.5 to 6.0 and maintained for 30 minutes. The reaction mass was cooled to 45° to 50° C. and maintained for 30 minutes. The reaction mass was cooled to 25° to 35° C. and maintained for 30 minutes. The product was filtered and washed with methanol (50 ml×2) afforded the formate salt of (S)-4-(4-(5-(aminomethyl)-2-oxo-oxazolidin-3-yl)phenyl)morpholin-3-one of Formula (JF).

### Example-16

### Preparation of oxalate salt of (S)-4-(4-(5-(aminomethyl)-2-oxo-oxazolidin-3-yl)phenyl) morpholin-3-one of Formula (JO)

100 g (S)-2-((2-oxo-3-(4-(3-oxo-morpholino)phenyl)oxazolidin-5-yl)methyl)isoindoline-1,3-dione of Formula (I), 1000 ml Methanol and 119 g methyl amine were added to RBF at 25° C. to 35° C. The reaction was heated to 60° C. to 65° C. for 1-2 hours. To the reaction mass oxalic acid (15 g) was added till to pH 5.5 to 6.0 and maintained for 30 minutes. The reaction mass was cooled to 45° to 50° C. and maintained for 30 minutes. The reaction mass was cooled to 25° to 35° C. and maintained for 30 minutes. The product was filtered and washed with methanol (50 ml×2) afforded the oxalate salt of (S)-4-(4-(5-(aminomethyl)-2-oxo-oxazolidin-3-yl)phenyl)morpholin-3-one of Formula (JO).

### Example-17

### Preparation of succinate salt of (S)-4-(4-(5-(aminomethyl)-2-oxo-oxazolidin-3-yl)phenyl) morpholin-3-one of Formula (JS)

100 g (S)-2-((2-oxo-3-(4-(3-oxo-morpholino)phenyl)oxazolidin-5-yl)methyl)isoindoline-1,3-dione of Formula (I), 1000 ml methanol and 119 g methyl amine were added to RBF at 25° C. to 35° C. The reaction was heated to 60° C. to 65° C. for 1-2 hours. To the reaction mass succinic acid (15 g) was added till to pH 5.5 to 6.0 and maintained for 30 minutes. The reaction mass was cooled to 45° to 50° C. and maintained for 30 minutes. The reaction mass was cooled to 25° to 35° C. and maintained for 30 minutes. The product was filtered and washed with methanol (50 ml×2) afforded succinate salt of (S)-4-(4-(5-(aminomethyl)-2-oxo-oxazolidin-3-yl)phenyl)morpholin-3-one of Formula (JS).

### Example-18

### Preparation of L (+)-mandelate salt of (S)-4-(4-(5-(Aminomethyl)-2-oxooxazolidin-3-yl)phenyl)morpholin-3-one of Formula (JM)

10 g (S)-2-((2-oxo-3-(4-(3-oxo-morpholino)phenyl)oxazolidin-5-yl)methyl)isoindoline-1,3-dione of Formula (I), 70 ml methanol and 60 ml monomethyl amine were added to RBF at 25° C. to 35° C. The reaction was heated to 60° C. to 65° C. for 1-2 hours. To the reaction mass L (+)-mandelic acid (3.6 g) was added and maintained for 30 minutes. The reaction mass was cooled to 45° to 50° C. and maintained for 30 minutes. The reaction mass was cooled to 25° to 35° C. and maintained for 30 minutes. The product was filtered and washed with methanol (50 ml×2) afforded the L(+)-mandelate salt of (S)-4-(4-(5-(aminomethyl)-2-oxo-oxazolidin-3-yl)phenyl)morpholin-3-one of Formula (JM).

### Example-20

### Preparation of Rivaroxaban

Thionyl chloride (10 ml) and 5-chlorothiophene-2-carboxylic acid (5 g) were added at 25° C. to 35° C. and heated to 65° C. to 70° C. and maintained for 60 minutes. Toluene (25 ml) was added and heated to 110° C. to 120° C. and excess thionyl chloride and toluene was distilled out. In another RBF L (+)-mandelate salt of (S)-4-(4-(5- and toluene was distilled out. In another RBF L (+)-mandelate salt of (S)-4-(4-(5-(aminomethyl)-2-oxo-oxazolidin-3-yl)phenyl)morpholin-3-one of Formula (JM) (10 g) was added and water (50 ml) and MDC (100 ml) was added at 25° C. to 35° C. and cooled to 0° C. to 5° C. followed by addition of sodium carbonate (4.5 g). To the reaction mass above prepared acid chloride solution was added at 0° C. to 5° C. and raised to 25° C. to 35° C. The reaction mass was filtered and washed with water (20 ml). The reaction mass was treated with 50% HCl solution (100 ml) at 50° C. to 60° C. and stirred for 30 minutes. The reaction mass was filtered and washed with water (20 ml) afforded as crude rivaroxaban of Formula (1).

10 g of crude rivaroxaban was treated with MDC (50 ml) at 25° C. to 35° C. and heated to 40° C. to 45° C. and stirred for 15 minutes. To the reaction mass methanol (50 ml) was added and stirred for 30 minutes. The product was filtered and washed with mixture of methanol (10 ml) afforded pure rivaroxaban.

While the present invention has been described in terms of its specific embodiments, certain modifications and equivalents will be apparent to those skilled in the art and are intended to be included within the scope of the present invention.

1. A process for the preparation of rivaroxaban of Formula (1) or its pharmaceutical acceptable salts, solvates, and hydrates thereof, 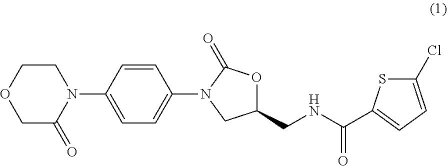 [SMILES: O=C(NC[C@H]1CN(c2ccc(N3CCOCC3=O)cc2)C(=O)O1)c1ccc(Cl)s1]
the process comprising:
i) reacting 4-(4-aminophenyl)morpholin-3-one compound of Formula (C) with (R)-glycidyl alkyl ester of Formula (E1), wherein R represents C₁-C₅ alkyl, in a suitable solvent to obtain (R)-2-hydroxy-3-(4-(3-oxomorpholino)phenylamino)alkyl ester of Formula (EE); 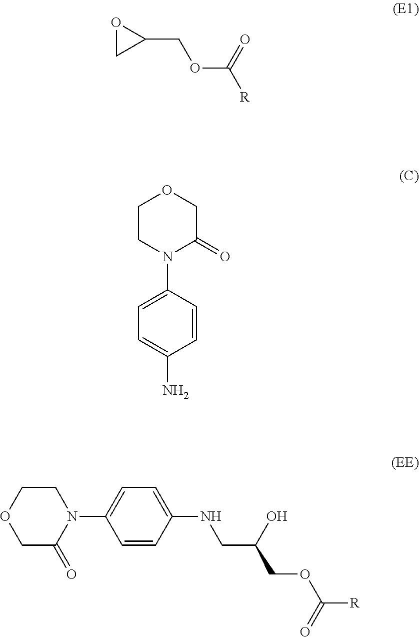
ii) reacting (R)-2-hydroxy-3-(4-(3-oxomorpholino)phenylamino)alkyl ester of Formula (EE) with a cyclizing agent in a suitable solvent, optionally in the presence of a catalyst to obtain (R)-(2-oxo-3-(4-(3-oxomorpholino)phenyl)oxazolidin-5-yl)alkyl ester of Formula (FF); 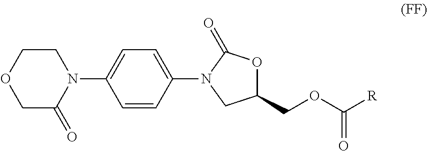
iii) reacting (R)-(2-oxo-3-(4-(3-oxomorpholino)phenyl)oxazolidin-5-yl)alkyl ester of Formula (FF) with a base to obtain (R)-4-(4-(5-(hydroxymethyl)-2-oxooxazolidin-3-yl)phenyl)morpholin-3-one of Formula (GG); 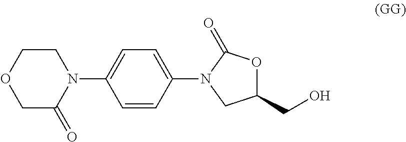 [SMILES: O=C1COCCN1c1ccc(N2C[C@H](CO)OC2=O)cc1]
iv) reacting (R)-4-(4-(5-(hydroxymethyl)-2-oxo-oxazolidin-3-yl)phenyl)morpholin-3-one of Formula (GG) with a compound of Formula (2), wherein R₁ represents C₁-C₅ alkyl or substituted or unsubstituted aryl, and X represents halide selected from Br, Cl, F or I, 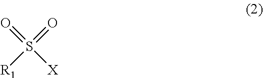
in the presence of a base to obtain a compound of Formula (HH); 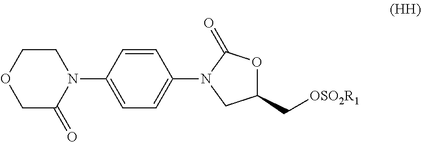
v) reacting the compound of Formula (HH) with phthalimide, optionally in the presence of a base to obtain (S)-2-((2-oxo-3-(4-(3-oxo-morpholino)phenyl)oxazolidin-5-yl)methyl)iso-indoline-1,3-dione of Formula (I); 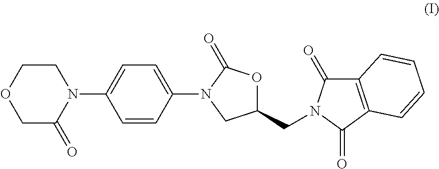 [SMILES: O=C1c2ccccc2C(=O)N1C[C@H]1CN(c2ccc(N3CCOCC3=O)cc2)C(=O)O1]
vi) reacting the (S)-2-((2-oxo-3-(4-(3-oxo-morpholino)phenyl)oxazolidin-5-yl)methyl)iso-indoline-1,3-dione of Formula (I) with a base in a suitable solvent to obtain (S)-4-(4-(5-(aminomethyl)-2-oxooxazolidin-3-yl)phenyl)morpholin-3-one (J); and 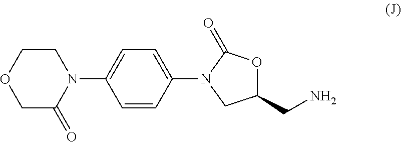 [SMILES: NC[C@H]1CN(c2ccc(N3CCOCC3=O)cc2)C(=O)O1]
vii) converting the (S)-4-(4-(5-(aminomethyl)-2-oxooxazolidin-3-yl)phenyl)morpholin-3-one (J) to rivaroxaban of Formula (I).

2. The process as claimed in claim 1, wherein R is butyl, R₁ is methyl or p-tolyl, and X is Cl.

3. The process as claimed in claim 1, wherein the cyclizing agent is N,N-carbonyldiimidazole (CDI) or phosgene.

4. The process as claimed in claim 1, wherein the catalyst comprises one or more of N,N-dimethylamino pyridine (DMAP), diisopropylamine (DIPA) and diisopropyethylamine (DIPEA).

5. The process as claimed in claim 1, wherein the base at step (iii) or step (v) comprises one or more of alkali or alkaline earth metal hydroxides, alkoxides, carbonates or bicarbonates selected from sodium hydroxide, potassium hydroxide, sodium methoxide, potassium tert-butoxide, sodium carbonate, potassium carbonate, and sodium bicarbonate.

6. The process as claimed in claim 1, wherein the base at step (iv) comprises one or more of alkali or alkaline earth metal hydroxides, alkoxides, carbonates or bicarbonates, or organic bases selected from sodium hydroxide, potassium hydroxide, sodium methoxide, potassium tert-butoxide, sodium carbonate, potassium carbonate, sodium bicarbonate, triethyl amine, diisopropyl amine, dimethyl amine, diisopropylethylamine, diisopropylmethyl amine, pyridine, piperidine, morpholine and N-methyl piperidine.

7. The process as claimed in claim 1, wherein the base at step (vi) comprises one or more of hydrazine hydrate, and C₁-C₅ amines.

8. The process as claimed in claim 1, wherein the suitable solvent at step (i) to (v) comprises one or more of C₁-C₅ alcohols, esters, ethers, nitrile, tetrahydrofuran (THF), water, halogenated solvents, dimethylformamide, dimethyl sulfoxide, sulfolane, or a mixture thereof.

9-13. (canceled)

14. The solid state form of (R)-2-hydroxy-3-(4-(3-oxomorpholino)phenylamino)propyl butyrate of Formula (E′) as claimed claim 54, which is characterized by one or more of the following properties:
i) a powder X-ray diffraction pattern substantially in accordance with FIG. 1;
ii) a powder X-ray diffraction pattern having peaks at about 6.8, 7.6, 8.4, 13.3, 17.5, 21.5, 22.7 and ±23.2±0.2 degrees 2-theta;
iii) an IR spectrum substantially in accordance with FIG. 2; and
iv) an IR spectrum having absorption bands at about 3371, 3332, 3043, 2954, 2870, 2833, 2358, 1870, 1842, 1728, 1691, 1629, 1608, 1572, 1490, 1465, 1450, 1392, 1371, 1350, 1330, 1301, 1259, 1230, 1188, 1118, 1024, 997, 921, 900, 831, 806, 756, 721, 690, 646, 603 and 549±2 cm-1.

15. (canceled)

16. The solid state form of (R)-(2-oxo-3-(4-(3-oxo-morpholino)phenyl)oxazolidin-5-yl)methyl butyrate of Formula (F′) as claimed in claim 54, which is characterized by one or more of the following properties:
i) a powder X-ray diffraction pattern substantially in accordance with FIG. 3;
ii) a powder X-ray diffraction pattern having peaks at about 5.5, 11.1, 14.9, 16.8, 18.5, 22.6, 23.4, 24.2 and 24.9±0.2 degrees 2-theta;
iii) an IR spectrum substantially in accordance with FIG. 4; and
iv) an IR spectrum having absorption bands at about 3458, 3292, 2974, 2954, 2877, 2358, 1869, 1732, 1651, 1517, 1481, 1386, 1352, 1336, 1315, 1294, 1180, 1128, 1089, 1045, 997, 925, 871, 821, 779, 752, 705, 665, 613, 538, 511 and 460±2 cm-1.

17-19. (canceled)

20. Use of the compounds of Formula (EE), Formula (FF), Formula (HH), Formula (E′), Formula (F′) as claimed in claim 54, in the preparation of rivaroxaban of Formula (I) or its pharmaceutically acceptable salts, solvates, and hydrates thereof.

21. (canceled)

22. A solid state form of acid addition salts of (S)-4-(4-(5-(aminomethyl)-2-oxo-oxazolidin-3-yl)phenyl)morpholin-3-one of Formula (J1), 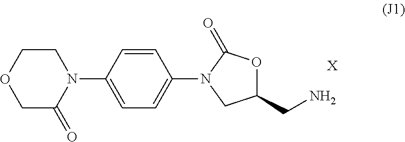
wherein X represents salts with inorganic acids or organic acids.

23. The salt of Formula (J1) as claimed in claim 22, wherein the inorganic acid may be selected from hydrochloric acid, hydrobromic acid, phosphoric acid, sulphuric acid and the organic acid may be selected from sulphonic acid, oxalic acid, formic acid, acetic acid, trifluoroacetic acid, propionic acid, maleic acid, succinic acid, fumaric acid, malic acid, citric acid, tartaric acid, lactic acid, benzoic acid, mandelic acid, methanesulphonic acid, ethanesulphonic acid, benzenesulphonic acid, toluenesulphonic acid, and naphthalenedisulphonic acid.

24. A process for the preparation of acid addition salts of (S)-4-(4-(5-(aminomethyl)-2-oxo-oxazolidin-3-yl)phenyl)morpholin-3-one of Formula (J1) as claimed in claim 22, 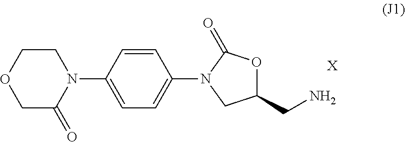
wherein X represents salts with inorganic acid or organic acid, the process comprising:
(i) providing a solution of (S)-4-(4-(5-(aminomethyl)-2-oxo-oxazolidin-3-yl) phenyl) morpholin-3-one of Formula (J) in a suitable solvent;
(ii) treating with an inorganic acid or an organic acid; and
(iii) obtaining the acid addition salt of (S)-4-(4-(5-(aminomethyl)-2-oxo-oxazolidin-3-yl)phenyl)morpholin-3-one of Formula (J1).

25. (canceled)

26. A solid state form of formate salt of (S)-4-(4-(5-(aminomethyl)-2-oxo-oxazolidin-3-yl)phenyl) morpholin-3-one of Formula (JF) as claimed in claim 23, 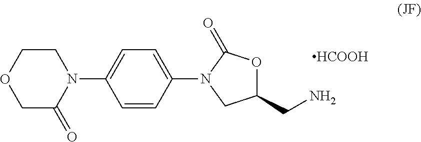 [SMILES: NC[C@H]1CN(c2ccc(N3CCOCC3=O)cc2)C(=O)O1 ; "·HCOOH"]
which is characterized by one or more of the following properties:
i) a powder X-ray diffraction pattern substantially in accordance with FIG. 13;
ii) a powder X-ray diffraction pattern having peaks at about 5.5, 11.1, 16.6, 16.8, 18.6, 20.3, 22.5, 24.9, 26.4, 27.1, 28.3, 30.5 and 34.1±0.2 degree 2θ;
iii) a differential scanning calorimetry (DSC) having peak at about 193.5° C.; and
iv) an IR spectrum having absorption bands at about 2872, 2783, 2688, 2355, 2191, 1917, 1743, 1724, 1693, 1660, 1647, 1552, 1517, 1477, 1431, 1413, 1342, 1282, 1228, 1186, 1138, 1120, 1097, 1060, 1022, 997, 960, 921, 864, 833, 783, 756, 711, 688, 596, 553, 464 and 428 cm⁻¹.

27. A solid state form of oxalate salt of (S)-4-(4-(5-(aminomethyl)-2-oxo-oxazolidin-3-yl)phenyl)morpholin-3-one of Formula (JO) as claimed in claim 23, 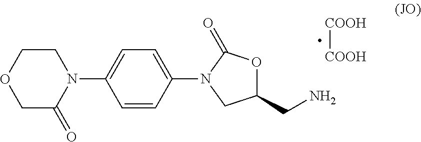
which is characterized by one or more, of the following properties:
i) a powder X-ray diffraction pattern substantially in accordance with FIG. 16; and
ii) a powder X-ray diffraction pattern having peaks at about 2.8, 5.1, 9.1, 14.2, 15.5, 16.9, 17.7, 18.0, 19.1, 19.7, 20.3, 20.5, 23.3, 24.2, 25.7, 26.8 and 28.6±0.2 degree 2θ.

28. A solid state form of succinate salt of (S)-4-(4-(5-(aminomethyl)-2-oxo-oxazolidin-3-yl)phenyl) morpholin-3-one of Formula (JS) as claimed in claim 23, 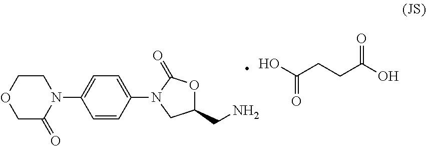 [SMILES: NC[C@H]1CN(c2ccc(N3CCOCC3=O)cc2)C(=O)O1 ; O=C(O)CCC(=O)O]
which is characterized by one or more of the following properties:
i) a powder X-ray diffraction pattern substantially in accordance with FIG. 17; and
ii) a powder X-ray diffraction pattern having peaks at about 2.5, 5.1, 5.7, 11.5, 14.5, 15.7, 16.2, 16.8, 17.5, 19.1, 19.6, 19.8, 20.5, 21.5, 21.8, 23.4, 24.6, 25.7, 26.2, 26.7 and 28.5, ±0.2 degree 2θ.

29. A solid state form of mandelate salt of (S)-4-(4-(5-(Aminomethyl)-2-oxooxazolidin-3-yl)phenyl)morpholin-3-one of Formula (JM) as claimed in claim 23, 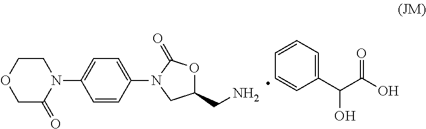 [SMILES: NC[C@H]1CN(c2ccc(N3CCOCC3=O)cc2)C(=O)O1 ; O=C(O)C(O)c1ccccc1]
which is characterized by one or more of the following properties:
1) a powder X-ray diffraction pattern substantially in accordance with FIG. 18; and
ii) a powder X-ray diffraction pattern having peaks at about 13.3, 14.6, 15.2, 15.9, 20.2, 20.9, 22.2, 24.6, 25.6 and 26.3±0.2 degree 2θ.

30. A process for the preparation of rivaroxaban of Formula (1) or its pharmaceutically acceptable salts, solvates, and hydrates thereof, the process comprising reacting (S)-4-(4-(5-(aminomethyl)-2-oxooxazolidin-3-yl)phenyl)morpholin-3-one of Formula (J) with 5-chlorothiophene-2-carboxylic acid chloride in a biphasic solvent system in the presence of a base to obtain the rivaroxaban.

31. The process as claimed claim 30, wherein the biphasic solvent system comprises mixture of solvents comprising one or more of water, hydrocarbon, nitrile, amide, alcohol, ketone, halogenated solvent and ester selected from toluene, xylene, ethylbenzene dimethyl formamide, dimethyl acetamide, acetonitrile, methanol, ethanol, propanol, isopropanol, butanol, acetone, methyl isobutyl ketone, methyl ethyl ketone, ethyl acetate, isopropyl acetate, and butyl acetate.

32. (canceled)

33. The process as claimed in claim 30, wherein the base comprises one or more of an organic or inorganic base; an organic base may be selected from one or more of diisopropylethylamine, diisopropylamine, triethylamine, diethylamine, pyridine, N-methyl piperidine, piperidine, morpholine, pyridine, DBU, and DABCO; and the inorganic base may be selected from one or more of sodium hydroxide, potassium hydroxide, lithium hydroxide, sodium carbonate, potassium carbonate, and cesium carbonate.

34-36. (canceled)

37. An improved process for the preparation of rivaroxaban of Formula (1) or pharmaceutically acceptable salts, solvates, and hydrates thereof, 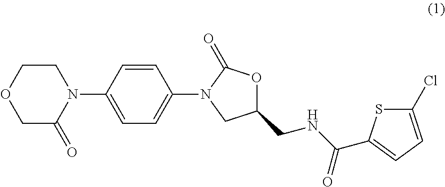 [SMILES: O=C(NC[C@H]1CN(c2ccc(N3CCOCC3=O)cc2)C(=O)O1)c1ccc(Cl)s1]
the process comprising:
i) reacting 4-(4-aminophenyl)morpholin-3-one of Formula (C) with (S)-2-(oxiran-2-yl methyl)isoindoline-1,3-dione of Formula (E) in a suitable solvent in the absence of a base to obtain (R)-2-(2-hydroxy-3-(4-(3-oxo-morpholino)phenylamino)propyl)isoindoline-1,3-dione of Formula (F);
ii) reacting the (R)-2-(2-hydroxy-3-(4-(3-oxo-morpholino)phenylamino)propyl) isoindoline-1,3-dione of Formula (F) with a cyclizing agent in a suitable solvent in the presence of a catalyst to obtain (S)-2-((2-oxo-3-(4-(3oxo-morpholino)phenyl) oxazolidin-5-yl)methyl) isoindoline-1,3-dione of Formula (I);
iii) reacting the (S)-2-((2-oxo-3-(4-(3-oxo-morpholino)phenyl)oxazolidin-5-yl)methyl)isoindoline-1,3-dione of Formula (G) in presence of base in a suitable solvent to obtain (S)-4-(4-(5-(aminomethyl)-2-oxooxazolidin-3-yl)phenyl)morpholin-3-one (J1); and
iv) reacting the (S)-4-(4-(5-(aminomethyl)-2-oxooxazolidin-3-yl)phenyl)morpholin-3-one of Formula (J) with 5-chlorothiophene-2-carboxylic acid chloride in a biphasic solvent system in the presence of a base to obtain rivaroxaban.

38-53. (canceled)

54. The compounds selected from:
(i) (R)-2-hydroxy-3-(4-(3-oxomorpholino)phenylamino)alkyl ester of Formula (EE), 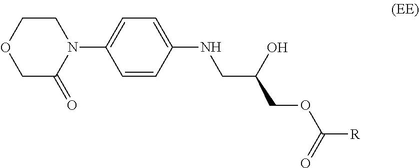
wherein R represents C₁-C₅ alkyl,
(ii) (R)-(2-oxo-3-(4-(3-oxomorpholino)phenyl)oxazolidin-5-yl)alkyl ester of Formula (FF), 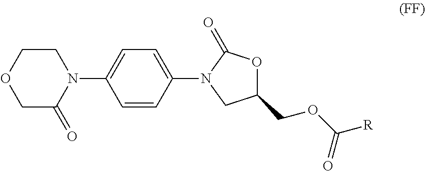
wherein R represents C₁-C₅ alkyl,
(iii) the compound of Formula (HH), 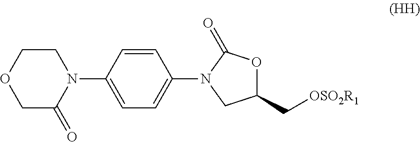
wherein R₁ represents C₁-C₅ alkyl or substituted or unsubstituted aryl, and X represents halide selected from Br, Cl, F or I,
(iv) (R)-2-hydroxy-3-(4-(3-oxo-morpholino)phenylamino)propyl butyrate of Formula (E′), 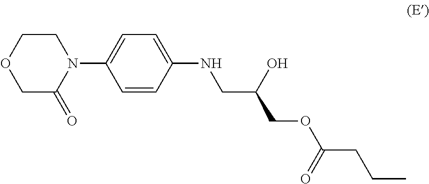 [SMILES: CCCC(=O)OC[C@H](O)CNc1ccc(N2CCOCC2=O)cc1]
(v) (R)-(2-oxo-3-(4-(3-oxo-morpholino)phenyl)oxazolidin-5-yl)methyl butyrate of Formula (F′). 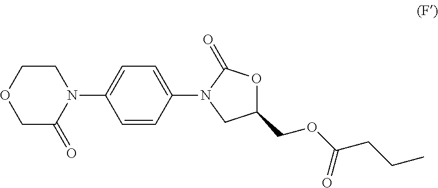 [SMILES: CCCC(=O)OC[C@H]1CN(c2ccc(N3CCOCC3=O)cc2)C(=O)O1]
